(19) United States
(12) Patent Application Publication
(10) Pub. No.: US 2009/0099136 A1
(43) Pub. Date: Apr. 16, 2009

(54) DIOXOLANE THYMINE PHOSPHORAMIDATES AS ANTI-HIV AGENTS

(75) Inventors: MICHAEL JOSEPH SOFIA, DOYLESTOWN, PA (US); PEIYUAN WANG, GLENROCK, NJ (US); SUGUNA H. RACHAKONDA, ROBBINSVILLE, NJ (US); JINFA DU, NEW HOPE, PA (US)

Correspondence Address: MERCHANT & GOULD PC, P.O. BOX 2903, MINNEAPOLIS, MN, 55402-0903, US

(73) Assignee: PHARMASSET, INC., PRINCETON, NJ (US)

(21) Appl. No.: 12/251,574

(22) Filed: Oct. 15, 2008

Related U.S. Application Data
(60) Provisional application No. 60/979,961, filed on Oct. 15, 2007.

(51) Int. Cl. C07F 9/6509; A61K 31/675; A61P 31/18

(52) U.S. Cl. 514/86; 544/232

(57) ABSTRACT

Disclosed are dioxolane thymine phosphoramidate compounds, compositions, and methods for using dioxolane thymine phosphoramidate compounds and compositions to treat viral infections, such as HIV infections.

### FIELD OF THE INVENTION

Embodiments of the invention are directed to compounds, methods, and compositions for use in the treatment of viral infections. More specifically embodiments of the invention are phosphoramidate-dioxolane-thymine compounds useful for the treatment of viral infections, such as HIV infections.

### BACKGROUND OF THE INVENTION

Acquired immune deficiency syndrome (AIDS) is a disease that severely compromises the human immune system, and that leads to death. The cause of AIDS has been determined to be the human immunodeficiency virus (HIV). To ameliorate suffering and to prolong the lives of infected hosts new compounds and methods of treating AIDS and attacking the HIV virus continue to be sought.

One area of active HIV research has been the study of dioxolane-thymine nucleosides. There has been a significant amount of research on 1,3-dioxolane nucleosides and their use to treat viral infections. U.S. Pat. Nos. 5,210,085; 5,276,151; 5,852,027; and 5,179,104 disclose 5-fluorocytosine-1,3-dioxolane nucleoside and nucleoside analogues for the treatment of viral infections.

An asymmetric process for the synthesis of dioxolane-thymine (DOT) is disclosed in U.S. Pat. No. 5,179,104, Jan. 12, 1993, C. K. Chu and R. F. Schinazi. Certain other nucleosides are also disclosed therein.

A molecular modeling study of the binding of DOT (C. K. Chu, V. Yadav, Y. H. Chong, and R. F. Schinazi, J. Med. Chem. (2005), 48, 3949-3952) with HIV reverse transcriptase demonstrated the importance of the dioxolane ring and its effect upon binding.

Nucleoside inhibitors of HIV reverse transcriptase can act either as a non-natural substrate that results in chain termination or as a competitive inhibitor which competes with nucleotide binding to the reverse transcriptase. To function as a chain terminator the nucleoside analog must be taken up by the cell and converted in vivo to a triphosphate to compete for the polymerase nucleotide binding site. This conversion to the triphosphate is commonly mediated by cellular kinases which impart additional structural requirements on a potential nucleoside polymerase inhibitor. Unfortunately, this limits the direct evaluation of nucleosides as inhibitors of HIV replication to cell-based assays capable of in situ phosphorylation.

In some cases, the biological activity of a nucleoside is hampered by its poor substrate characteristics for one or more of the kinases needed to convert it to the active triphosphate form. Formation of the monophosphate by a nucleoside kinase is generally viewed as the rate limiting step of the three phosphorylation events. To circumvent the need for the initial phosphorylation step in the metabolism of a nucleoside to the active triphosphate analog, the preparation of stable phosphate prodrugs has been reported. Nucleoside phosphoramidate prodrugs have been shown to be precursors of the active nucleoside triphosphate and to inhibit viral replication when administered to viral infected whole cells (McGuigan, C., et al., J. Med. Chem., 1996, 39, 1748-1753; Valette, G., et al., J. Med. Chem., 1996, 39, 1981-1990; Balzarini, J., et al., Proc. Natl. Acad Sci USA, 1996, 93, 7295-7299; Siddiqui, A. Q., et al., J. Med. Chem., 1999, 42, 4122-4128; Eisenberg, E. J., et al., Nucleosides, Nucleotides and Nucleic Acids, 2001, 20, 1091-1098; Lee, W. A., et al., Antimicrobial Agents and Chemotherapy, 2005, 49, 1898).

Also limiting the utility of nucleosides as viable therapeutic agents are their sometimes poor physicochemical and pharmacokinetic properties. These poor properties can limit the intestinal absorption of an agent and limit uptake into the target tissue or cell. To improve their properties prodrugs of nucleosides have been employed. It has been demonstrated that preparation of nucleoside phosphoramidates improves the systemic absorption of a nucleoside and furthermore, the phosphoramidate moiety of these “pronucleotides” is masked with neutral lipophilic groups to obtain a suitable partition coefficient to optimize uptake and transport into the cell dramatically enhancing the intracellular concentration of the nucleoside monophosphate analog relative to administering the parent nucleoside alone. Enzyme-mediated hydrolysis of the phosphate ester moiety produces a nucleoside monophosphate wherein the rate limiting initial phosphorylation is unnecessary.

It has been suggested that one of the limitations of DOT as an HIV agent is that it is a poor substrate for the first kinase in route to generation of the active triphosphate metabolite.

There has been interest in analogues of DOT. U.S. Patent application publication 2005/0209196, Sep. 22, 2005, by C. K. Chu and R. F. Schinazi describes a series of substituted DOT analogues, some of which may show improved properties. A generic phosphoramidate-dioxolane-thymine structure is disclosed but no specific compounds are described.

In an article, Bioorg. Med. Chem., 2006, 14, 2178-2189, Y. Liang, J. Narayanasamy, R. F. Schinazi, and C. K. Chu, a series of phosphoramidate-dioxolane-thymine compounds are described. Some of the compounds described showed potent anti-HIV activity.

The preceding references and all other references cited in the present specification are hereby incorporated herein by reference.

It is an object of embodiments of the invention to provide a compound, method, and composition for the treatment or prevention of HIV infection in a host. It is a further object of embodiments of the invention to provide a compound, method, and composition for the treatment or prevention of HIV when the host is a human, or when the host is an animal.

### SUMMARY OF THE INVENTION

Embodiments of the invention comprise compounds and mixtures useful for treating viral infections. It has been found that certain dioxolane nucleosides show improved inhibitory activity against HIV. Therefore a method for the treatment or prevention of HIV infection in a host, and in particular, a human, is provided that includes administering an effective amount of a dioxolane thymine phosphoramidate nucleotide.

In one embodiment of the invention the active compound is of formula I:

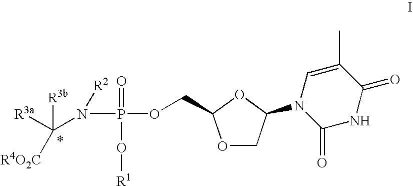

wherein:

R¹ is hydrogen, n-alkyl, branched alkyl, substituted or unsubstituted cycloalkyl, or aryl, which includes, but is not limited to, phenyl or naphthyl,
where phenyl or naphthyl is optionally substituted with at least one of C₁₋₆ alkyl, C₂₋₆ alkenyl, C₂₋₆ alkynyl, C₁₋₆ alkoxy, F, Cl, Br, I, nitro, cyano, C₁₋₆ haloalkyl, —N(R^{1′})₂, C₁₋₆ acylamino, —NHSO₂C₁₋₆ alkyl, —SO₂N(R^{1′})₂, COR^{1″}, and —SO₂C₁₋₆ alkyl,
   where R^{1′} is independently hydrogen or alkyl, which includes, but is not limited to, C₁₋₂₀ alkyl, C₁₋₁₀ alkyl, or C₁₋₆ alkyl, and R^{1∝1} is —OR′ or —N(R^{1′})₂;

R² is hydrogen, C₁₋₁₀ alkyl, either R^{3a} and R² or R^{3b} and R² together are (CH₂)ₙ so as to form a cyclic ring that includes the adjoining N and C atoms, C(O)CR^{3a}R^{3b}NHR¹, where n is 2 to 4 and R¹, R^{3a}, and R^{3b} are as defined herein;

R^{3a} and R^{3b} are
(i) independently selected from hydrogen, C₁₋₁₂ alkyl (particularly when the alkyl is an amino acid residue), —(CH₂)_{c}(NR^{3′})₂, C₁₋₆ hydroxyalkyl, —CH₂SH, —(CH₂)₂S(O)_{d}Me, —(CH₂)₃NHC(═NH)NH₂, (1H-indol-3-yl)methyl, (1H-imidazol-4-yl)methyl, —(CH₂)_{c}COR^{3″}, aryl or aryl C₁₋₃ alkyl, said aryl groups optionally substituted with a group selected from hydroxyl, C₁₋₁₀ alkyl, C₁₋₆ alkoxy, halogen, nitro or cyano,
   where c is 1 to 6, d is 0 to 2, and e is 0 to 3 and
   R^{3′} is independently hydrogen or C₁₋₆ alkyl and R^{3″} is —)R^{3′} or —N(R^{3′})₂,
(ii) R^{3a} and R^{3b} both are C₁₋₆ alkyl,
(iii) R^{3a} and R^{3b} together are (CH₂)_{f} so as to form a spiro ring,
   where f is 3 to 5,
(iv) R^{3a} is hydrogen and R^{3b} and R² together are (CH₂)ₙ so as to form a cyclic ring that includes the adjoining N and C atoms,
   where n is 2 to 4,
(v) R^{3b} is hydrogen and R^{3a} and R² together are (CH₂)ₙ so as to form a cyclic ring that includes the adjoining N and C atoms,
   where n is 2 to 4,
(vi) R^{3a} is H and R^{3b} is independently selected from H, CH₃, CH(CH₃)₂, CH₂CH(CH₃)₂, CH(CH₃)CH₂CH₃, CH₂Ph, (1H-indol-3-yl)methyl, (1H-imidazol-4-yl)methyl, —CH₂CH₂SCH₃, CH₂CO₂H, CH₂C(O)NH₂, CH₂CH₂COOH, CH₂CH₂C(O)NH₂, CH₂CH₂CH₂CH₂NH₂, —CH₂CH₂CH₂NHC(NH)NH₂, CH₂OH, CH(OH)CH₃, CH₂((4′-OH)-Ph), or CH₂SH, or
(vii) R^{3a} is CH₃, CH(CH₃)₂, CH₂CH(CH₃)₂, CH(CH₃)CH₂CH₃, CH₂Ph, CH₂-indol-3-yl, —CH₂CH₂SCH₃, CH₂CO₂H, CH₂C(O)NH₂, CH₂CH₂COOH, CH₂CH₂C(O)NH₂, CH₂CH₂CH₂CH₂NH₂, —CH₂CH₂CH₂NHC(NH)NH₂, CH₂-imidazol-4-yl, CH₂-imidazol-4-yl, CH₂OH, CH(OH)CH₃, CH₂((4′-OH)-Ph), or CH₂SH and R^{3b} is H; and

R⁴ is hydrogen, C₁₋₁₀ alkyl, C₁₋₁₀ alkyl optionally substituted with a lower alkyl, alkoxy, substituted or unsubstituted cycloalkyl, halogen, C₁₋₁₀ haloalkyl, or substituted or unsubstituted aryl;

with the proviso that that the active compound represented by formula I is not selected from the group consisting of:

| | | | | | |
|---|---|---|---|---|---|
| (1) | R1 = 1-Napth | R2 = H | R3a = H | R3b = Me | R4 = CH₂Ph; |
| (2) | R1 = 4-Br-Ph | R2 = H | R3a = H | R3b = Me | R4 = Me; |
| (3) | R1 = 2,4-diCl-Ph | R2 = H | R3a = H | R3b = Me | R4 = Me; |
| (4) | R1 = 4-F-Ph | R2 = H | R3a = H | R3b = Me | R4 = Me; |
| (5) | R1 = 4-Cl-Ph | R2 = H | R3a = H | R3b = Me | R4 = Me; |
| (6) | R1 = 1-Napth | R2 = H | R3a = H | R3b = Me | R4 = Me; |
| (7) | R1 = Ph | R2 = H | R3a = H | R3b = Me | R4 = Me; |
| (8) | R1 = Ph | R2 = H | R3a = H | R3b = iPr | R4 = Me; |
| (9) | R1 = Ph | R2 = H | R3a = H | R3b = H | R4 = CH₃; |
| (10) | R1 = Ph | R2 = H | R3a = Me | R3b = Me | R4 = Me; |
| (11) | R1 = Ph | R2 = H | R3a = Me | R3b = H | R4 = Me; |
| (12) | R1 = Ph | R2 = H | R3a = H | R3b = CH₂Ph | R4 = Me; |
| (13) | R1 = Ph | R2 = H | R3a = CH₂Ph | R3b = H | R4 = Me; |
| (14) | R1 = Ph | R2 = H | R3a = iPr | R3b = H | R4 = Me; |
| (15) | R1 = Ph | R2 = H | R3a = H | R3b = Me | R4 = t-Bu; |
| (16) | R1 = Ph | R2 = H | R3a = H | R3b = Me | R4 = CH₂Ph; |
| (17) | R1 = 4-Me-Ph | R2 = H | R3a = H | R3b = Me | R4 = CH₃; |
| (18) | R1 = 4-Propyl-Ph | R2 = H | R3a = H | R3b = Me | R4 = Me; |
| (19) | R1 = 4-Neopent-Ph | R2 = H | R3a = H | R3b = Me | R4 = Me; |
| (20) | R1 = 4-MeO-Ph | R2 = H | R3a = H | R3b = Me | R4 = Me; |
| (21) | R1 = 4-CN-Ph | R2 = H | R3a = H | R3b = Me | R4 = Me; |
| (22) | R1 = 4-Br-Ph | R2 = H | R3a = H | R3b = Me | R4 = CH₂Ph; |
| (23) | R1 = 2-Cl-Ph | R2 = H | R3a = H | R3b = Me | R4 = Me; |
| (24) | R1 = 4-Cl-Ph | R2 = H | R3a = H | R3b = Me | R4 = CH₂Ph; |
| (25) | R1 = 2-Allyl-Ph | R2 = H | R3a = H | R3b = Me | R4 = Me; |
| (26) | R1 = 1-Napth | R2 = H | R3a = Me | R3b = Me | R4 = Me; |
| (27) | R1 = C₁₆H₃₃O(CH₂)₃ | R2 = H | R3a = H | R3b = H | R4 = Me; |
| (28) | R1 = C₁₆H₃₃O(CH₂)₃ | R2 = H | R3a = H | R3b = Me | R4 = Me; |
| (29) | R1 = C₁₆H₃₃O(CH₂)₃ | R2 = H | R3a = H | R3b = iPr | R4 = Me; |
| (30) | R1 = C₁₈H₃₇O(CH₂)₂ | R2 = H | R3a = H | R3b = Me | R4 = Me; and |
| (31) | R1 = Oleyl | R2 = H | R3a = H | R3b = Me | R4 = Me. |

The asterisk (*) in formula I is intended to show that the carbon is chiral when R^{3a} and R^{3b} are different substituents.

Embodiments of the present invention provide a compound, method, and composition for treating an HIV infection in a host comprising administering a therapeutically effective amount of at least one compound as described in the present application.

Embodiments of the present invention provide a compound, method, and composition for preventing an HIV infection in a host comprising administering a therapeutically effective amount of at least one compound as described in the present application.

### DETAILED DESCRIPTION OF THE INVENTION

Applicants have discovered that dioxolane phosphoramidate nucleosides, and in particular, dioxolane thymine, show improved inhibitory activity against HIV. Therefore, a method for the treatment or prevention of a host, and in particular, a human, infected with HIV is provided that includes administering an effective amount of a dioxolane nucleoside.

Embodiments of the present invention provide a compound, method, and composition for treating an HIV infection in a host comprising administering a therapeutically effective amount of at least one compound as described in the present application.

Embodiments of the present invention provide a compound, method, and composition for preventing an HIV infection in a host comprising administering a therapeutically effective amount of at least one compound as described in the present application.

In another aspect, embodiments of the invention provide a pharmaceutical formulation comprising a compound of the invention in combination with a pharmaceutically acceptable carrier or excipient.

In another aspect, embodiments of the invention provide a method and composition for treating or preventing HIV infection in a host comprising administering to the host a combination comprising at least one compound of the invention and at least one further therapeutic agent.

In one embodiment of the invention the active compound is of formula I:

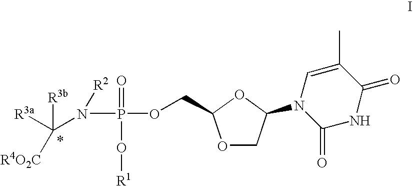

wherein:

R¹ is hydrogen, n-alkyl, branched alkyl, substituted or unsubstituted cycloalkyl, or aryl, which includes, but is not limited to, phenyl or naphthyl,
where phenyl or naphthyl is optionally substituted with at least one of C₁₋₆ alkyl, C₂₋₆ alkenyl, C₂₋₆ alkynyl, C₁₋₆ alkoxy, F, Cl, Br, I, nitro, cyano, C₁₋₆ haloalkyl, —N(R^{1′})₂, C₁₋₆ acylamino, —NHSO₂C₁₋₆ alkyl, —SO₂N(R^{1′})₂, COR^{1″}, and —SO₂C₁₋₆ alkyl,
   where R^{1′} is independently hydrogen or alkyl, which includes, but is not limited to, C₁₋₂₀ alkyl, C₁₋₁₀ alkyl, or C₁₋₆ alkyl, and R^{1″} is —OR′ or —N(R^{1′})₂;

R² is hydrogen, C₁₋₁₀ alkyl, either R^{3a} and R² or R^{3b} and R² together are (CH₂)ₙ so as to form a cyclic ring that includes the adjoining N and C atoms, C(O)CR^{3a}R^{3b}NHR¹, where n is 2 to 4 and R¹, R^{3a}, and R^{3b} are as defined herein;

R^{3a} and R^{3b} are
(i) independently selected from hydrogen, C₁₋₁₂ alkyl (particularly when the alkyl is an amino acid residue), —(CH₂)_{c}(NR^{3′})₂, C₁₋₆ hydroxyalkyl, —CH₂SH, —(CH₂)₂S(O)_{d}Me, —(CH₂)₃NHC(═NH)NH₂, (1H-indol-3-yl)methyl, (1H-imidazol-4-yl)methyl, —(CH₂)ₑCOR^{3″}, aryl or aryl C₁₋₃ alkyl, said aryl groups optionally substituted with a group selected from hydroxyl, C₁₋₁₀ alkyl, C₁₋₆ alkoxy, halogen, nitro or cyano,
   where c is 1 to 6, d is 0 to 2, and e is 0 to 3 and
   R^{3′} is independently hydrogen or C₁₋₆ alkyl and R^{3″} is —OR^{3′} or —N(R^{3′})₂,
(ii) R^{3a} and R^{3b} both are C₁₋₆ alkyl,
(iii) R^{3a} and R^{3b} together are (CH₂)_{f} so as to form a spiro ring,
   where f is 3 to 5,
(iv) R^{3a} is hydrogen and R^{3b} and R² together are (CH₂)ₙ so as to form a cyclic ring that includes the adjoining N and C atoms,
   where n is 2 to 4,
(v) R^{3b} is hydrogen and R^{3a} and R² together are (CH₂)ₙ so as to form a cyclic ring that includes the adjoining N and C atoms,
   where n is 2 to 4,
(vi) R^{3a} is H and R^{3b} is independently selected from H, CH₃, CH(CH₃)₂, CH₂CH(CH₃)₂, CH(CH₃)CH₂CH₃, CH₂Ph, (1H-indol-3-yl)methyl, (1H-imidazol-4-yl)methyl, —CH₂CH₂SCH₃, CH₂CO₂H, CH₂C(O)NH₂, CH₂CH₂COOH, CH₂CH₂C(O)NH₂, CH₂CH₂CH₂CH₂NH₂, —CH₂CH₂CH₂NHC(NH)NH₂, CH₂OH, CH(OH)CH₃, CH₂((4′-OH)-Ph), or CH₂SH, or
(vii) R^{3a} is CH₃, CH(CH₃)₂, CH₂CH(CH₃)₂, CH(CH₃)CH₂CH₃, CH₂Ph, CH₂-indol-3-yl, —CH₂CH₂SCH₃, CH₂CO₂H, CH₂C(O)NH₂, CH₂CH₂COOH, CH₂CH₂C(O)NH₂, CH₂CH₂CH₂CH₂NH₂, —CH₂CH₂CH₂NHC(NH)NH₂, CH₂-imidazol-4-yl, CH₂OH, CH(OH)CH₃, CH₂((4′-OH)-Ph), or CH₂SH and R^{3b} is H; and

R⁴ is hydrogen, C₁₋₁₀ alkyl, C₁₋₁₀ alkyl optionally substituted with a lower alkyl, alkoxy, substituted or unsubstituted cycloalkyl, halogen, C₁₋₁₀ haloalkyl, or substituted or unsubstituted aryl;

with the proviso that that the active compound represented by formula I is not selected from the group consisting of:

| | | | | | |
|---|---|---|---|---|---|
| (1) | R1 = 1-Napth | R2 = H | R3a = H | R3b = Me | R4 = CH₂Ph; |
| (2) | R1 = 4-Br-Ph | R2 = H | R3a = H | R3b = Me | R4 = Me; |
| (3) | R1 = 2,4-diCl-Ph | R2 = H | R3a = H | R3b = Me | R4 = Me; |
| (4) | R1 = 4-F-Ph | R2 = H | R3a = H | R3b = Me | R4 = Me; |
| (5) | R1 = 4-Cl-Ph | R2 = H | R3a = H | R3b = Me | R4 = Me; |
| (6) | R1 = 1-Napth | R2 = H | R3a = H | R3b = Me | R4 = Me; |
| (7) | R1 = Ph | R2 = H | R3a = H | R3b = Me | R4 = Me; and |
| (8) | R1 = Ph | R2 = H | R3a = H | R3b = iPr | R4 = Me. |
| (9) | R1 = Ph | R2 = H | R3a = H | R3b = H | R4 = CH₃; |
| (10) | R1 = Ph | R2 = H | R3a = Me | R3b = Me | R4 = Me; |
| (11) | R1 = Ph | R2 = H | R3a = Me | R3b = H | R4 = Me; |
| (12) | R1 = Ph | R2 = H | R3a = H | R3b = CH₂Ph | R4 = Me; |
| (13) | R1 = Ph | R2 = H | R3a = CH₂Ph | R3b = H | R4 = Me; |
| (14) | R1 = Ph | R2 = H | R3a = iPr | R3b = H | R4 = Me; |
| (15) | R1 = Ph | R2 = H | R3a = H | R3b = Me | R4 = t-Bu; |
| (16) | R1 = Ph | R2 = H | R3a = H | R3b = Me | R4 = CH₂Ph; |
| (17) | R1 = 4-Me-Ph | R2 = H | R3a = H | R3b = Me | R4 = CH₃; |
| (18) | R1 = 4-Propyl-Ph | R2 = H | R3a = H | R3b = Me | R4 = Me; |
| (19) | R1 = 4-Neopent-Ph | R2 = H | R3a = H | R3b = Me | R4 = Me; |
| (20) | R1 = 4-MeO-Ph | R2 = H | R3a = H | R3b = Me | R4 = Me; |
| (21) | R1 = 4-CN-Ph | R2 = H | R3a = H | R3b = Me | R4 = Me; |
| (22) | R1 = 4-Br-Ph | R2 = H | R3a = H | R3b = Me | R4 = CH₂Ph; |
| (23) | R1 = 2-Cl-Ph | R2 = H | R3a = H | R3b = Me | R4 = Me; |
| (24) | R1 = 4-Cl-Ph | R2 = H | R3a = H | R3b = Me | R4 = CH₂Ph; |
| (25) | R1 = 2-Allyl-Ph | R2 = H | R3a = H | R3b = Me | R4 = Me; |
| (26) | R1 = 1-Napth | R2 = H | R3a = Me | R3b = Me | R4 = Me; |
| (27) | R1 = C₁₆H₃₃O(CH₂)₃ | R2 = H | R3a = H | R3b = H | R4 = Me; |
| (28) | R1 = C₁₆H₃₃O(CH₂)₃ | R2 = H | R3a = H | R3b = Me | R4 = Me; |
| (29) | R1 = C₁₆H₃₃O(CH₂)₃ | R2 = H | R3a = H | R3b = iPr | R4 = Me; |
| (30) | R1 = C₁₈H₃₇O(CH₂)₂ | R2 = H | R3a = H | R3b = Me | R4 = Me; and |
| (31) | R1 = Oleyl | R2 = H | R3a = H | R3b = Me | R4 = Me. |

The asterisk (*) in formula I is intended to show that the carbon is chiral when R^{3a} and R^{3b} are different substituents.

In one embodiment of the invention, the active compound is of formula I, its pharmaceutically acceptable salt or prodrugs thereof, wherein:
i) R¹ is 3,4-dichlorophenyl;
ii) R² is H;
iii) R^{3a} is H;
iv) R^{3b} is methyl; and
v) R⁴ is methyl.

In one embodiment of the invention, the active compound is of formula I, its pharmaceutically acceptable salt or prodrugs thereof, wherein:
i) R¹ is phenyl;
ii) R² is H;
iii) R^{3a} is H;
iv) R^{3b} is methyl; and
v) R⁴ is butyl.

In one embodiment of the invention, the active compound is of formula I, its pharmaceutically acceptable salt or prodrugs thereof, wherein:
i) R¹ is bromophenyl;
ii) R² is H;
iii) R^{3a} is H;
iv) R^{3b} is isopropyl; and
v) R⁴ is methyl.

In one embodiment of the invention, the active compound is of formula I, its pharmaceutically acceptable salt or prodrugs thereof, wherein:
i) R¹ is phenyl;
ii) R² is H;
iii) R^{3a} is H;
iv) R^{3b} is benzyl; and
v) R⁴ is ethyl.

In one embodiment of the invention, the active compound is of formula I, its pharmaceutically acceptable salt or prodrugs thereof, wherein:
i) R¹ is naphthyl;
ii) R² is H;
iii) R^{3a} is H;
iv) R^{3b} is methyl; and
v) R⁴ is ethyl.

In one embodiment of the invention, the active compound is of formula I, its pharmaceutically acceptable salt or prodrugs thereof, wherein:
i) R¹ is phenyl;
ii) R² is H;
iii) R^{3a} is H;
iv) R^{3b} is methyl; and
v) R⁴ is ethyl.

In one embodiment of the invention, the active compound is of formula I, its pharmaceutically acceptable salt or prodrugs thereof, wherein:
i) R¹ is phenyl;
ii) R² is H;
iii) R^{3a} is H;
iv) R^{3b} is methyl; and
v) R⁴ is 2-butyl.

In one embodiment of the invention, the active compound is of formula I, its pharmaceutically acceptable salt or prodrugs thereof, wherein:
i) R¹ is phenyl;
ii) R² is H;
iii) R^{3a} is H;
iv) R^{3b} is methyl; and
v) R⁴ is isopropyl.

In one embodiment of the invention, the active compound is of formula I, its pharmaceutically acceptable salt or prodrugs thereof, wherein:
i) R¹ is phenyl;
ii) R² is H;
iii) R^{3a} is methyl;
iv) R^{3b} is methyl; and
v) R⁴ is benzyl.

In one embodiment of the invention, the active compound is of formula I, its pharmaceutically acceptable salt or prodrugs thereof, wherein:
i) R¹ is phenyl;
ii) R² is H;
iii) R^{3a} is H;
iv) R^{3b} is H; and
v) R⁴ is benzyl.

In one embodiment of the invention, the active compound is of formula I, its pharmaceutically acceptable salt or prodrugs thereof, wherein:
i) R¹ is methoxyphenyl;
ii) R² is H;
iii) R^{3a} is H;
iv) R^{3b} is methyl; and
v) R⁴ is benzyl.

In one embodiment of the invention, the active compound is of formula I, its pharmaceutically acceptable salt or prodrugs thereof, wherein:
i) R¹ is phenyl;
ii) R² is H;
iii) R^{3a} is H;
iv) R^{3b} is H; and
v) R⁴ is ethyl.

In one embodiment of the invention, the active compound is of formula I, its pharmaceutically acceptable salt or prodrugs thereof, wherein:
i) R¹ is phenyl;
ii) R^{3a} is H;
iii) R₂ and R_{3b} connect N and Cα-carbon via —(CH₂)₃—; and
iv) R⁴ is methyl.

In other embodiments of the invention, the active compound is one of the compounds listed in Table 1, its pharmaceutically acceptable salts or prodrugs thereof.

TABLE 1

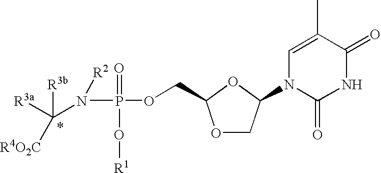

| R¹ | R² | R^{3a} | R^{3b} | R⁴ |
|---|---|---|---|---|
| Ph | H | H | Me | pentyl |
| Ph | H | H | Me | hexyl |
| Ph | H | H | Me | 4-F-Bn |
| 4-Cl-Ph | H | H | Me | Et |
| 4-Cl-Ph | H | H | Me | i-Pr |
| 4-Cl-Ph | H | H | Me | n-Bu |
| 4-Cl-Ph | H | H | Me | benzyl (Bn) |
| 2-Cl-Ph | H | H | Me | i-Pr |
| 2-Cl-Ph | H | H | Me | n-Bu |
| 2-Cl-Ph | H | H | Me | Bn |
| 4-Br-Ph | H | H | Me | Et |
| 4-Br-Ph | H | H | Me | i-Pr |
| 4-Br-Ph | H | H | Me | n-Bu |
| 4-Br-Ph | H | H | Me | hexyl |
| 4-Br-Ph | H | H | Me | propyl |
| 4-Br-Ph | H | H | Me | pentyl |
| 4-Br-Ph | H | H | Me | 2-Bu |
| 4-Br-Ph | H | H | Me | cyclo-hex |
| 4-Br-Ph | H | H | Me | t-Bu |
| 4-F-Ph | H | H | Me | Et |
| 4-F-Ph | H | H | Me | i-Pr |
| 4-F-Ph | H | H | Me | n-Bu |
| 4-F-Ph | H | H | Me | Bn |
| 2,4-di-Cl-Ph | H | H | Me | Et |
| 2,4-di-Cl-Ph | H | H | Me | i-Pr |
| 2,4-di-Cl-Ph | H | H | Me | n-Bu |
| 2,4-di-Cl-Ph | H | H | Me | Bn |
| 3,4-di-Cl-Ph | H | H | Me | Et |
| 3,4-di-Cl-Ph | H | H | Me | i-Pr |
| 3,4-di-Cl-Ph | H | H | Me | n-Bu |
| 3,4-di-Cl-Ph | H | H | Me | Bn |
| 4-MeO-Ph | H | H | Me | i-Pr |
| 4-MeO-Ph | H | H | Me | n-Bu |
| 4-Me-Ph | H | H | Me | i-Pr |
| 4-Me-Ph | H | H | Me | n-Bu |
| 4-Me-Ph | H | H | Me | Bn |
| Ph | H | H | i-Bu (Leu) | Me |
| Ph | H | H | 3-indolyl-CH₂- | Me |
| | | | (Trp) | |
| Ph | H | H | Sec-Butyl (Ile) | Me |
| Ph | H | H | Methylmercapto- | Me |
| | | | Et (Met) | |
| 4-Br-Ph | H | H | i-Butyl (Leu) | Me |
| 4-Br-Ph | H | H | i-Bu (Leu) | Et |
| 4-Br-Ph | H | H | i-Bu (Leu) | i-Pr |
| 4-Br-Ph | H | H | i-Bu (Leu) | n-Bu |
| 4-Br-Ph | H | H | i-Bu (Leu) | Bn |
| 4-Br-Ph | H | Me | H | Me |
| 4-Br-Ph | H | Me | H | n-Bu |
| 4-Br-Ph | H | Me | H | Bn |
| 4-F-Ph | H | H | i-Bu (Leu) | Me |
| 4-F-Ph | H | H | i-Bu (Leu) | Bn |
| 4-F-Ph | H | Me | H | Me |
| 4-F-Ph | H | Me | H | Bn |
| 4-Cl-Ph | H | H | i-Bu (Leu) | Me |
| 4-Cl-Ph | H | H | i-Bu (Leu) | Bn |
| 4-Cl-Ph | H | Me | H | Me |
| 4-Cl-Ph | H | Me | H | Bn |
| Ph | H | H | Me | Cyc-hex |
| Ph | H | H | Me | Cyc-pent |
| 4-Br-Ph | H | H | Me | Cyc-pent |
| 4-Br-Ph | H | H | i-Bu (Leu) | Cyc-pent |
| 4-F-Ph | H | H | Et | Cyc-hex |
| 4-Cl-Ph | H | H | Et | Cyc-hex |
| 4-Br-Ph | H | H | Et | Cyc-hex |
| Ph | H | H | Et | Cyc-hex |
| 4-F-Ph | H | H | i-Bu(Leu) | Cyc-hex |
| 4-Cl-Ph | H | H | i-Bu(Leu) | Cyc-hex |
| 4-Br-Ph | H | H | i-Bu(Leu) | Cyc-hex |
| Ph | H | H | i-Bu (Leu) | Cyc-hex |
| 4-MeO-Ph | H | H | Me | Cyc-hex |
| 4-F-Ph | H | H | Me | Cyc-hexyl |
| 4-F-Ph | H | H | Me | Cyc-pentyl |
| 4-F-Ph | H | H | Me | Cyc-butyl |
| 4-F-Ph | H | H | Me | Cyc-propylmethyl |
| 4-Br-Ph | H | H | Me | Cyc-pentyl |
| 4-Br-Ph | H | H | Me | Cyc-butyl |
| 4-Br-Ph | H | H | Me | Cyc-propylmethyl |
| 4-Cl-Ph | H | H | Me | Cyc-hexyl |
| 4-Cl-Ph | H | H | Me | Cyc-pentyl |
| 4-Cl-Ph | H | H | Me | Cyc-butyl |
| 4-Cl-Ph | H | H | Me | Cyc-propylmethyl |
| Ph | H | H | Me | Cyc-butyl |
| Ph | H | H | Me | Cyc-propylmethyl |
| Ph | H | H | Me | —CH₂CF₃ |
| 4-F-Ph | H | H | Me | —CH₂CF₃ |
| 4-Br-Ph | H | H | Me | —CH₂CF₃ |
| Ph | H | H | Me | (1,2-Dimethyl-propyl) |
| Ph | H | H | Me | (1-Methyl-butyl) |
| Ph | H | H | Me | (1-Methyl-pentyl) |
| Ph | H | H | Me | (1-Ethyl-propyl) |
| Ph | H | H | Me | (1,3-Dimethyl-butyl)- |
| Ph | H | H | Me | (1,2-Dimethyl-butyl) |
| Ph | H | H | Me | (1-Cyclopropyl-ethyl) |
| Ph | H | H | Me | (1-Methyl- |
| | | | | cyclopropylmethyl) |
| Ph | H | H | Me | (2-Methyl- |
| | | | | cyclopropylmethyl) |
| Ph | H | H | Me | Cyclobutylmethyl- |
| Ph | H | H | Me | Cyclopentylmethyl- |
| Ph | H | H | Me | 1-Cyclopentyl-ethyl |
| Ph | H | H | Me | Cyclohexyylmethyl- |
| Ph | H | H | Me | 1-Cyclohexyl-ethyl |
| Ph | H | H | Me | 1-Phenyl-ethyl |
| Ph | H | H | Me | 1-(4-Fluoro-phenyl)-ethyl |
| Ph | H | H | i-Bu (Leu) | Cyclopropyl-methyl |
| Ph | H | Me | H | cyclopropyl-methyl |
| Ph | H | Me | H | 4-F-Ph-CH₂ |
| Ph | H | Me | H | CH₂Ph |
| 4-FPh | H | Me | Me | Me |
| Ph | H | # | # | Me |

# R^{3a} and R^{3b} linked with —(CH₂)₂—.

### Definitions

The term “DOT,” as used herein, refers to the compound dioxolane thymine shown below:

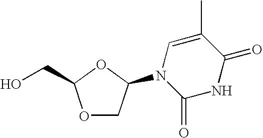

The term “TEA,” as used herein, refers to the compound triethylamine.

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the first definition provided in the Summary of the Invention.

The terms “optional” or “optionally” as used herein means that a subsequently described event or circumstance may but need not occur, and that the description includes instances where the event or circumstance occurs and instances in which it does not. For example, “optional bond” means that the bond may or may not be present, and that the description includes single, double, or triple bonds.

The term “independently” is used herein to indicate that a variable is applied in any one instance without regard to the presence or absence of a variable having that same or a different definition within the same compound. Thus, in a compound in which R appears twice and is defined as “independently carbon or nitrogen”, both R's can be carbon, both R's can be nitrogen, or one R′ can be carbon and the other nitrogen.

The term “alkenyl” refers to an unsubstituted hydrocarbon chain radical having from 2 to 10 carbon atoms having one or two olefinic double bonds, preferably one olefinic double bond. The term “C_{2-N} alkenyl” refers to an alkenyl comprising 2 to N carbon atoms, where N is an integer having the following values: 3, 4, 5, 6, 7, 8, 9, or 10. The term “C₂₋₆ alkenyl” refers to an alkenyl comprising 2 to 6 carbon atoms and is synonymous with the term “lower alkenyl.” The term “C₂₋₁₀ alkenyl” refers to an alkenyl comprising 2 to 10 carbon atoms. The term “C₂₋₄ alkenyl” refers to an alkenyl comprising 2 to 4 carbon atoms. Examples include, but are not limited to, vinyl, 1-propenyl, 2-propenyl(allyl) or 2-butenyl(crotyl).

The term “halogenated alkenyl” refers to an alkenyl comprising at least one of F, Cl, Br, and I.

The term “alkyl” refers to an unsubstituted or substituted, unbranched or branched chain, saturated, monovalent hydrocarbon residue containing 1 to 30 carbon atoms. The term “C_{1-M} alkyl” refers to an alkyl comprising 1 to M carbon atoms, where M is an integer having the following values: 2, 3, 4, 5, 6, 7, 8, 9, 10, 11, 12, 13, 14, 15, 16, 17, 18, 19, 20, 21, 22, 23, 24, 25, 26, 27, 28, 29, or 30. The term “C₁₋₄ alkyl” refers to an alkyl containing 1 to 4 carbon atoms. The term “C₁₋₆ alkyl” refers to an alkyl comprising 1 to 6 carbon atoms and is synonymous with the term “lower alkyl.” “C₁₋₂₀ alkyl” as used herein refers to an alkyl comprising 1 to 20 carbon atoms. “C₁₋₁₀ alkyl” as used herein refers to an alkyl comprising 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, octyl, etc. The alkyl can be substituted by a substituted or an unsubstituted cycloalkyl, an aryl, or a heteroaryl. The term (ar)alkyl or (heteroaryl)alkyl indicate the alkyl group is optionally substituted by an aryl or a heteroaryl group respectively.

The term “halogenated alkyl” (or “haloalkyl”) refers to an unbranched or branched chain alkyl comprising at least one of F, Cl, Br, and I. The term “C₁₋₃ haloalkyl” refers to a haloalkyl comprising 1 to 3 carbons and at least one of F, Cl, Br, and I. The term “halogenated lower alkyl” refers to a haloalkyl comprising 1 to 6 carbon atoms and at least one of F, Cl, Br, and I. Examples include, but are not limited to, fluoromethyl, chloromethyl, bromomethyl, iodomethyl, difluoromethyl, dichloromethyl, dibromomethyl, diiodomethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-difluoroethyl, 2,2-dichloroethyl, 2,2-dibromomethyl, 2-2-diiodomethyl, 3-fluoropropyl, 3-chloropropyl, 3-bromopropyl, 2,2,2-trifluoroethyl or 1,1,2,2,2-pentafluoroethyl.

The term “alkynyl” refers to an unbranched or branched hydrocarbon chain radical having from 2 to 10 carbon atoms, preferably 2 to 5 carbon atoms, and having one triple bond. The term “C_{2-N} alkynyl” refers to an alkynyl comprising 2 to N carbon atoms, where N is an integer having the following values: 3, 4, 5, 6, 7, 8, 9, or 10. The term “C₂₋₆ alkynyl” refers to an alkynyl comprising 2 to 6 carbon atoms and is synonymous with the term “lower alkynyl.” The term “C C₂₋₄ alkynyl” refers to an alkynyl comprising 2 to 4 carbon atoms. The term “C₂₋₁₀ alkynyl” refers to an alkynyl comprising 2 to 10 carbons. Examples include, but are limited to, ethynyl, 1-propynyl, 2-propynyl, 1-butynyl, 2-butynyl or 3-butynyl.

The term “halogenated alkynyl” refers to an unbranched or branched hydrocarbon chain radical having from 2 to 10 carbon atoms, preferably 2 to 5 carbon atoms, and having one triple bond and at least one of F, Cl, Br, and I.

The term “cycloalkyl” refers to a saturated carbocyclic ring comprising 3 to 8 carbon atoms, i.e. cyclopropyl, cyclobutyl, cyclopentyl, cyclohexyl, cycloheptyl or cyclooctyl. The term “C₃₋₇ cycloalkyl” as used herein refers to a cycloalkyl comprising 3 to 7 carbons in the carbocyclic ring. The cycloalkyl can be substituted with one or more moieties selected from among hydroxyl, amino, alkylamino, arylamino, alkoxy, aryloxy, nitro, halogen (F, Cl, Br, or I), cyano, sulfonic acid, sulfate, phosphonic acid, phosphate, or phosphonate, either unprotected, or protected as necessary, as known to those skilled in the art, for example, as taught in T. W. Greene and P. G. M. Wuts, “Protective Groups in Organic Synthesis,” 3rd ed., John Wiley & Sons, 1999.

The term “alkoxy” refers to an —O-alkyl group, wherein alkyl is as defined above. Examples include, but are not limited to, methoxy, ethoxy, n-propyloxy, i-propyloxy, n-butyloxy, i-butyloxy, t-butyloxy. The term “C₁₋₆ alkoxy” refers to an alkoxy comprising 1 to 6 carbon atoms and is synonymous with the term “lower alkoxy.” “C₁₋₁₀ alkoxy” refers to an —O-alkyl wherein alkyl is C₁₋₁₀.

The term “halogenated alkoxy” refers to an —O-alkyl group in which the alkyl group comprises at least one of F, Cl, Br, and I.

The term “halogenated lower alkoxy” refers to an —O-(lower alkyl) group in which the lower alkyl group comprises at least one of F, Cl, Br, and I.

The term “amino acid” includes naturally occurring and synthetic α, β, γ, or δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. In a preferred embodiment, the amino acid is in the L-configuration. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleucinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. When the term amino acid is used, it is considered to be a specific and independent disclosure of each of the esters of α, β, γ, or δ glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine in the D and L-configurations.

The terms “alkylamino” or “arylamino” refer to an amino group that has one or two alkyl or aryl substituents, respectively.

The term “protected,” as used herein and unless otherwise defined, refers to a group that is added to an oxygen, nitrogen, or phosphorus atom to prevent its further reaction or for other purposes. A wide variety of oxygen and nitrogen protecting groups are known to those skilled in the art of organic synthesis. Non-limiting examples include: C(O)-alkyl, C(O)Ph, C(O)aryl, CH₃, CH₂-alkyl, CH₂-alkenyl, CH₂Ph, CH₂-aryl, CH₂O-alkyl, CH₂O-aryl, SO₂-alkyl, SO₂-aryl, tert-butyldimethylsilyl, tert-butyldiphenylsilyl, and 1,3-(1,1,3,3-tetraisopropyldisiloxanylidene).

The term “aryl,” as used herein, and unless otherwise specified, refers to substituted or unsubstituted phenyl (Ph), biphenyl, or naphthyl, preferably the term aryl refers to substituted or unsubstituted phenyl. The aryl group can be substituted with one or more moieties selected from among hydroxyl, amino, alkylamino, arylamino, alkoxy, aryloxy, nitro, halogen (F, Cl, Br, or I), cyano, sulfonic acid, sulfate, phosphonic acid, phosphate, or phosphonate, either unprotected, or protected as necessary, as known to those skilled in the art, for example, as taught in T. W. Greene and P. G. M. Wuts, “Protective Groups in Organic Synthesis,” 3rd ed., John Wiley & Sons, 1999.

The terms “alkaryl” or “alkylaryl” refer to an alkyl group with an aryl substituent. The terms “aralkyl” or “arylalkyl” refer to an aryl group with an alkyl substituent.

The term “halo,” as used herein, includes chloro, bromo, iodo and fluoro.

The term “acyl” refers to a substituent containing a carbonyl moiety and a non-carbonyl moiety. The carbonyl moiety contains a double-bond between the carbonyl carbon and a heteroatom, where the heteroatom is selected from among O, N and S. When the heteroatom is N, the N is substituted by a lower alkyl. The non-carbonyl moiety is selected from straight, branched, or cyclic alkyl, which includes, but is not limited to, a straight, branched, or cyclic C₁₋₂₀ alkyl, C₁₋₁₀ alkyl, or lower alkyl; alkoxyalkyl, including methoxymethyl; aralkyl, including benzyl; aryloxyalkyl, such as phenoxymethyl; or aryl, including phenyl optionally substituted with halogen (F, Cl, Br, I), hydroxyl, C₁ to C₄ alkyl, or C₁ to C₄ alkoxy, sulfonate esters, such as alkyl or aralkyl sulphonyl, including methanesulfonyl, the mono, di or triphosphate ester, trityl or monomethoxytrityl, substituted benzyl, trialkylsilyl (e.g. dimethyl-t-butylsilyl) or diphenylmethylsilyl. When at least one aryl group is present in the non-carbonyl moiety, it is preferred that the aryl group comprises a phenyl group.

The term “lower acyl” refers to an acyl group in which the non-carbonyl moiety is lower alkyl.

The term “heteroatom,” as used herein, refers to oxygen, sulfur, nitrogen, and phosphorus.

The terms “heteroaryl” or “heteroaromatic,” as used herein, refers to an aromatic ring that includes one sulfur, oxygen, nitrogen, or phosphorus atom within the ring.

The term “heterocyclic,” as used herein, refers to a nonaromatic cyclic group wherein there is at least one heteroatom, such as oxygen, sulfur, nitrogen, or phosphorus in the ring.

The term “host,” as used herein, refers to a unicellular or multicellular organism in which the virus can replicate, including but not limited to cell lines and animals, and preferably a human. Alternatively, the host can be carrying a part of the viral genome, whose replication or function can be altered by the compounds of the present invention. The term host specifically refers to infected cells, cells transfected with all or part of the viral genome and animals, in particular, primates (including but not limited to chimpanzees) and humans. In most animal application of the present invention, the host is a human patient. Veterinary applications, in certain indication, however, are clearly anticipated by the present invention (such as chimpanzees).

The term “pharmaceutically acceptable salt or prodrug” is used throughout the specification to describe any pharmaceutically acceptable form (such as an ester, phosphate ester, salt of an ester or a related group) of a nucleoside compound which, upon administration to a patient, provides the nucleoside compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The compounds of the invention possess antiviral activity against HIV, or are metabolized to a compound that exhibits such activity.

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed including but not limited to, sulfate, nitrate, bicarbonate, and carbonate salts.

Pharmaceutically acceptable salts may be obtained using standard procedures well known in the art. for example by reacting a sufficiently basic compound such as an amine with a suitable acid, affording a physiologically acceptable anion. Alkali metal (e.g. sodium, potassium, or lithium) or alkaline earth metal (e.g. calcium or magnesium) salts of carboxylic acids can also be made.

In another embodiment for the treatment of HIV infection, the active compound or its prodrug or pharmaceutically acceptable salt can be administered in combination or alternation with another antiviral agent, such as another active anti-HIV agent, including but not limited to those of the formulae above, others listed below or known in the art. In general, in combination therapy, effective dosages of two or more agents are administered together, whereas during alternation therapy, an effective dosage of each agent is administered serially. The dosage will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

Nonlimiting examples of antiviral agents that can be used in combination with the compounds disclosed herein include the following: Invirase®, Fortovase®, Norvir®, Crixivan®, Viracept®, Agenerase®, Kaletra®, Retrovir®, Epivir®, Combivir®, Triazivir®, Ziagen®, Hivid®, Videx®, Didex® EC, Zerit®, Viread®, Covincil™, Viramune®, Rescriptor®, Sustiva®, Droxia®, Fuzeon®, Atazanavir®, Proleukin®, Remune®, Procrit®, Darunavir®, and Serostim®.

### Experimental Results

Phosphoramidate compounds can be prepared by condensation of a DOT (5) with a suitably substituted phosphochloridate compound 4 (Scheme 1), which can be prepared as follows. A suitably substituted hydroxyl compound R¹OH, such as a suitably substituted phenol, can be reacted with phosphorus oxychloride (1) to afford an aryloxy phosphorodichloridate 2 (see Example 1) which is subsequently treated with an acid addition salt of an N—R²-substituted α-amino acid R⁴-ester in the presence of TEA to afford an aryloxy phosphorochloridate 4. This arylalkoxy-amino acid phosphoramidate is reacted with DOT to provide the product I (for procedure see, e.g., C. McGuigan et al. Antiviral Res. 1002, 17:311-321; D. Curley et al Antiviral Res. 1990, 14:345-356; McGuigan et al. Antiviral Chem. Chemother. 1990 1(2):107-113). In many cases, the desired product is readily separated from the starting material using column chromatography on silica gel. The synthetic scheme is summarized in Scheme 1 below.

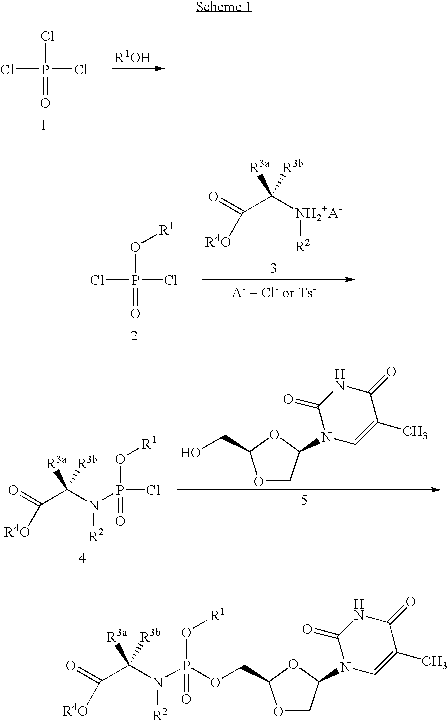

The following examples are intended to illustrate, but are not intended to limit embodiments of invention.

### EXAMPLE 1

### General Procedure for Preparation of phosphorodichloridates

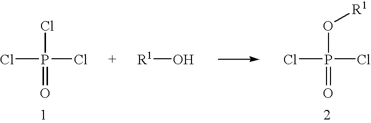

A solution of the suitably substituted phenol R¹—OH (1 eq) and triethylamine (1 eq.) in anhydrous ether was added dropwise to a stirred solution of phosphoryl trichloride 1 (1 eq) at 0° C. over a period of 3 hours under nitrogen. Then the temperature was warmed to room temperature, and the reaction was stirred overnight. The triethylamine salt was removed with suction filtration and the filtrate concentrated in vacuo to dryness to afford 2 as an oil which was used without further purification.

### EXAMPLE 2

### General Procedure for Preparation of phosphorochloridates

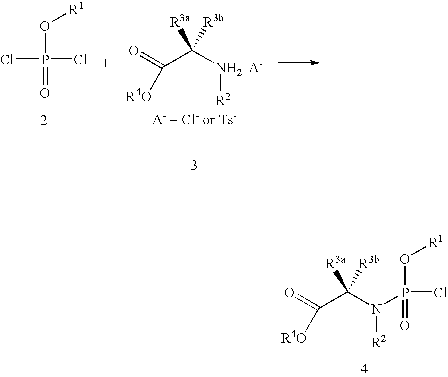

A solution of triethylamine (2 eq) in anhydrous dichloromethane was added dropwise to a solution of aryloxy-phosphodichloridate 2 (1 eq) and the appropriate amino ester 3 (1 eq) in anhydrous dichloromethane with vigorous stirring at −78° C. over a period of 30 to 120 minutes. The reaction temperature was then allowed to warm to room temperature and stirred over night. Solvent was removed. The residue was washed with ethyl ether and filtered. The filtrate was dried under reduced pressure to give 4.

### EXAMPLE 3

### General Procedures for DOT phosphoramidate Derivatives

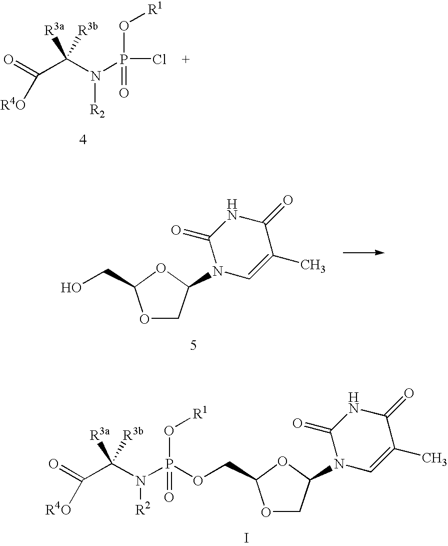

A solution of the appropriate phosphorochloridate 4 (6.5 equivalents) in anhydrous THF was added to a mixture of DOT 5 (1 equivalent) and N-methylimidazole (8 equivalents) in anhydrous THF with vigorous stirring at room temperature and the reaction mixture was stirred overnight. The solvent was removed in vacuo and the crude compound was purified by column chromatography and/or preparative thin layer chromatography to give I.

### EXAMPLE 4

### Preparation of D-dioxolane-thymine 5′-(4-bromophenyl methoxy-valyl phosphate)

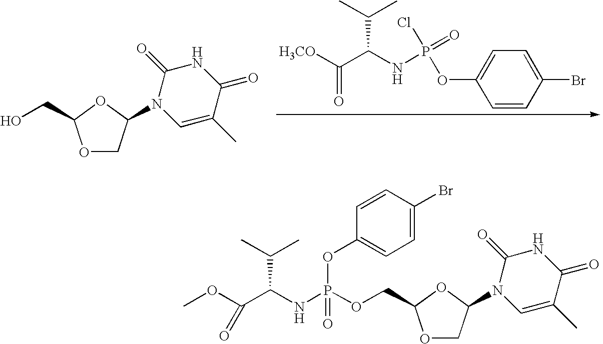

4-Bromophenyl methoxy-valyl phosphorochloridate (1 g, 3.4 eq ) dissolved in 3 mL of THF was added to a mixture of DOT (0.1 g, 1 eq) and N-methylimidazole (0.35 g, 6 eq) in 3 mL THF with vigorous stirring at room temperature, then the reaction was stirred overnight. Solvent was removed under reduced pressure, and the residue was further purified by pre-HPLC to give the product as a white solid (21.9 mg, 8.4%). ¹H NMR (DMSO-d₆) δ 0.68-0.81 (m, 6H), 1.67 (d, J=8.0 Hz, 3H), 1.83-1.88 (m, 1H), 3.44-3.48 (m, 1H), 3.54 (d, J=2.0 Hz, 3H), 4.08-4.26 (m, 4H), 5.12 (s, 1H), 5.99 (t, J=12.0 Hz, 1H), 6.26 (d, J=2.0 Hz, 1H), 7.10(d, J=4.0 Hz, 2H), 7.38(d, J=4.0 Hz, 1H), 7.51(d, J=4.6 Hz, 2H), 11.31 (s, 1H); MS, m/e 576 (M+1)⁺.

### EXAMPLE 5

### Preparation of D-dioxolane-thymine 5′-(phenyl ethoxy-alanyl phosphate)

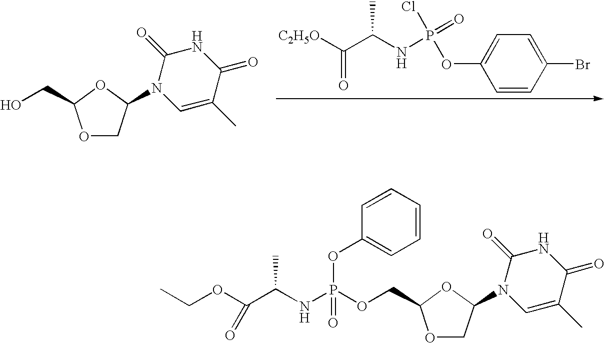

Phenyl ethoxy-alanyl phosphorochloridate (0.52 g, 2.03 eq ) dissolved in 10 mL of THF was added to a mixture of DOT (0.2 g, 1 eq) and N-methylimidazole (0.29 g, 4.05 eq) in 10 mL THF with vigorous stirring at room temperature, then the reaction was stirred overnight. Solvent was removed under reduced pressure, and the residue was further purified by pre-HPLC to give the product as a white solid (95 mg, 22.4%). 1H NMR (DMSO-d6) δ 1.11-1.23 (m, 6H), 1.70 (d, 3H), 3.75-3.82 (m, 1H), 4.01-4.06 (m, 2H), 4.11-4.30 (m, 6H), 5.13-5.16 (d, 1H), 6.01-6.11 (m, 1H), 6.28-6.31 (m, 1H), 7.14-7.18 (m, 3H), 7.33-7.38 (m, 2H), 7.41-7.46 (m, 1H), 11.35 (s, 1H); MS, m/e 484.1 (M+1)+.

### EXAMPLE 6

### Preparation of D-dioxolane-thymine 5′-(phenyl n-butoxy-alanyl phosphate)

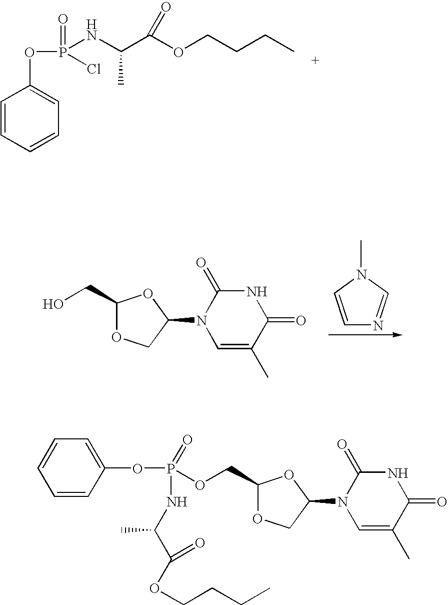

Phenyl n-butoxy-alanyl phosphorochloridate (695 mg, 2.17 mmol) dissolved in 10 mL of anhydrous THF was added to a mixture of DOT (200 mg, 0.88 mmol) and N-methylimidazole (250 mg, 3 mmol) in 15 mL THF with vigorous stirring at room temperature, then the reaction was stirred overnight. Solvent was removed under reduced pressure, and the residue was further purified by pre-HPLC under neutral conditions to give the product (62.52 mg) as a solid. 1H NMR (DMSO-d6) δ 0.82-0.83(m, 3H), 1.23-1.36 (m, 5H), 1.44-1.51(m, 2H), 1.68-1.73 (m, 3H), 3.69-3.80 (m, 1H), 3.91-3.99 (m, 2H), 4.08-4.27 (m, 4H), 5.12 (d, J=14.8 Hz, 1H), 6.00-6.09 (m, 1H), 6.27 (d, J=5.2 Hz, 1H), 7.09-7.14 (m, 3H), 7.32-7.35(m, 2H), 7.41(d, J=14 Hz, 1H), 11.31 (s, 1H); MS, m/e 512.3 (M+1)+.

### EXAMPLE 7

### Preparation of D-dioxolane-thymine 5′-(phenyl sec-butoxy-alanyl phosphate)

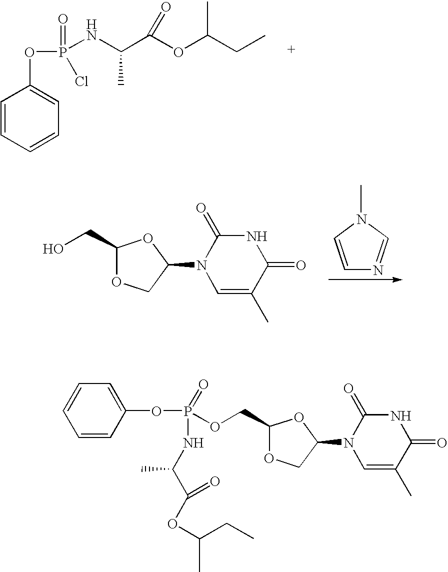

Phenyl sec-butoxy-alanyl phosphorochloridate (500 mg, 1.57 mmol) dissolved in 10 mL of anhydrous THF was added to a mixture of DOT (200 mg, 0.88 mmol) and N-methylimidazole (300 mg, 3.7 mmol) in 15 mL THF with vigorous stirring at room temperature, then the reaction was stirred overnight. Solvent was removed under reduced pressure, and the residue was further purified by pre-HPLC under neutral conditions to give the product (92.85 mg, yield: 21%) as a solid. 1H NMR (DMSO-d6) δ 0.93 (m, 6 H), 1.13-1.19 (m, 3 H), 1.64 (d, J=6.8 Hz, 3 H), 1.75-1.78 (m, 1 H), 3.67-3.78 (m, 3 H), 4.06-4.23 (m, 4 H),5.07 (d, J=15.6 Hz, 1H), 5.98-6.04 (m, 1 H), 6.22-6.24 (m, 1 H), 7.09-7.12 (m, 3 H), 7.26-7.35 (m, 2 H), 7.37 (d, J=13.6 Hz, 1 H), 11.31 (d, 1 H); MS, m/e 512.4 (M+1)+.

### EXAMPLE 8

### Preparation of D-dioxolane-thymine 5′-(phenyl isoproxy-alanyl phosphate)

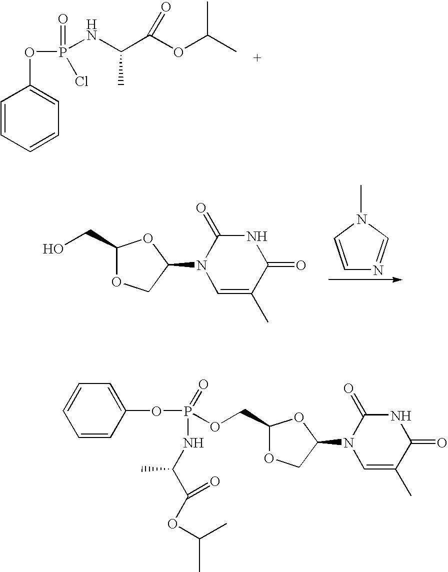

Phenyl isoproxy-alanyl phosphorochloridate (800 mg, 2.6 mmol) dissolved in 10 mL of anhydrous THF was added to a mixture of DOT (200 mg, 0.88 mmol) and N-methylimidazole (300 mg, 3.7 mmol) in 15 mL THF with vigorous stirring at room temperature, then the reaction was stirred overnight. Solvent was removed under reduced pressure, and the residue was further purified by pre-HPLC under neutral conditions to give the product (40.88 mg, yield: 9.3%) as a solid. 1H NMR(DMSO-d6) δ 1.12-1.21 (m, 9 H), 1.70-1.71(m, 3 H), 3.67-3.81(m, 1 H), 4.11-4.15(m, 1 H), 4.20-4.29 (m, 3 H), 4.81-4.85 (m, 1 H),5.13(d, J=15.2 Hz, 1H), 6.00-6.06 (m, 1 H), 6.27-6.30 (m, 1 H), 7.14-7.18 (m, 3 H), 7.32-7.41 (m, 2 H), 7.43 (d, J=13.6 Hz, 1 H), 11.31 (d, 1 H); MS, m/e 498.4 (M+1)+.

### EXAMPLE 9

### Preparation of D-dioxolane-thymine 5′-(phenyl ethoxy-phenylalanyl phosphate)

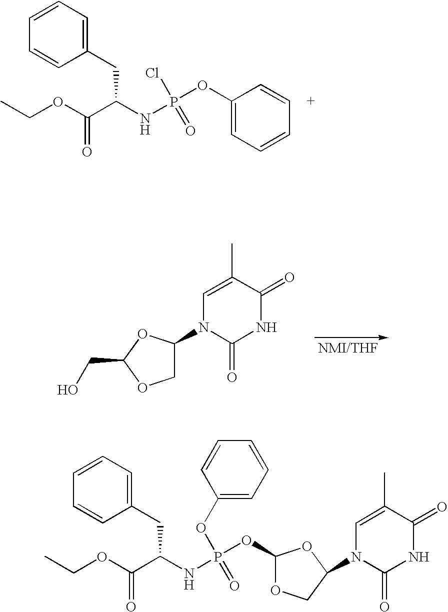

To a solution of DOT (200 mg, 0.9 mmol) and NMI (300 mg, 3.7 mmol) dissolved in anhydrous THF (15 ml) was added phenyl ethoxy-phenylalanyl phosphorochloridate in THF, and the mixture was stirred at room temperature overnight. Solvent was removed under reduced pressure, and the residue was further purified by pre-HPLC to give the product as a white solid (97.35 mg, Yield: 19%). 1H NMR(400 MHz, DMSO-d6):δ=1.04-1.11 (s, 3H), 1.72-1.74 (s, 3H), 2.85 (m, 1H), 2.87(m, 1H), 3.95-4.02 (s, 4H), 4.15-4.19 (s, 2H), 4.28 (s, 1H), 5.10 (s, 1H), 6.30 (m, 1H), 6.34-6.35 (m, 1H), 7.06-7.07 (s, 2H), 7.18-7.20 (s, 2H), 7.23-7.41 (s, 6H), 7.46 (s, 1H), 11.40 (m, 1H). MS, m/e 559.97 (M+1)+.

### EXAMPLE 10

### Preparation of D-dioxolane-thymine 5′-(4-methoxyphenyl benzyloxy-alanyl phosphate)

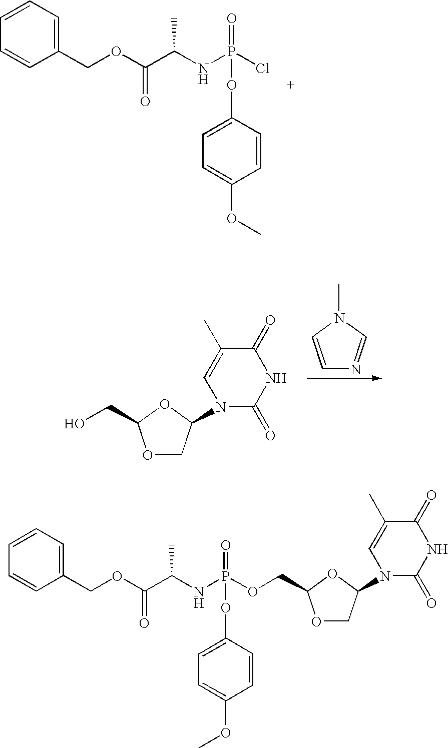

4-Methoxyphenyl benzyloxy-alanyl phosphorochloridate (0.7 g, 2.45 eq ) dissolved in 10 mL of THF was added to a mixture of DOT (0.17 g, 1 eq) and N-methylimidazole (0.30 g, 4.89 eq) in 10 mL THF with vigorous stirring at room temperature, then the reaction was stirred overnight. Solvent was removed under reduced pressure, and the residue was further purified by pre-HPLC to give the product as a white solid (49.4 mg, 11.5 %). 1H NMR (DMSO-d6) δ 1.18-1.26 (m, 3H), 1.69-1.71 (m, 3H), 3.71(s.3H) 3.80-3.91 (m,1H), 4.08-4.28 (m, 4H), 5.05-5.13 (m, 3H), 6.01-6.15 (m, 1H), 6.28-6.30 (d, 1H), 6.83-6.88 (m, 2H), 7.04-7.08 (m, 2H), 7.34 (s, 5H), 7.40-7.45 (m, 1H), 11.35 (s, 1H); MS, m/e 576.2 (M+1)+.

### EXAMPLE 11

### Preparation of D-dioxolane-thymine 5′-(naphthalenyl ethoxy-alanyl phosphate)

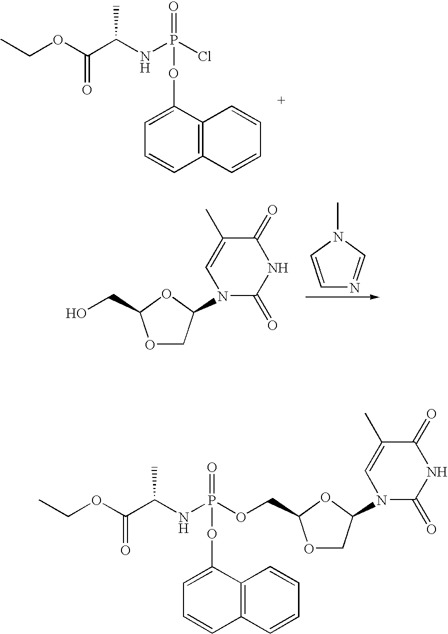

Naphthalenyl benzyloxy-alanyl phosphorochloridate (0.62 g, 2.07 eq ) dissolved in 10 mL of THF was added to a mixture of DOT (0.2 g, 1 eq) and N-methylimidazole (0.30 g, 4.15 eq) in 10 mL THF with vigorous stirring at room temperature, then the reaction was stirred overnight. Solvent was removed under reduced pressure, and the residue was further purified by pre-HPLC to give the product as a white solid (48.5 mg, 10.4%). 1H NMR (DMSO-d6) δ 1.06-1.14 (m, 3H), 1.19-1.25 (m, 3H),1.52-1.60 (d, 3H), 3.80-4.03 (d, 3H), 4.10-4.31 (m, 4H), 5.16-5.20 (d, 1H), 6.28-6.33 (m, 2H), 7.41-7.57 (m, 5H), 7.72-7.75 (m, 1H), 7.92-7.96 (m, 1H), 8.05-8.11 (m, 1H), 11.35 (d,1H); MS, m/e 534.2 (M+1)+.

### EXAMPLE 12

### Preparation of D-dioxolane-thymine 5′-(3,4-dichlorophenyl methoxy-alanyl phosphate)

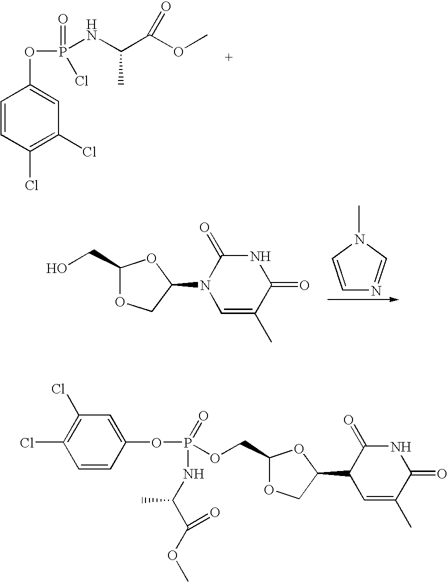

3,4-dichlorophenyl methoxy-alanyl phosphorochloridate (807 mg, 2.3 mmol) dissolved in 10 mL of anhydrous THF was added to a mixture of DOT (200 mg, 0.88 mmol) and N-methylimidazole (300 mg, 3.7 mmol) in 15 mL THF with vigorous stirring at room temperature, then the reaction was stirred overnight. Solvent was removed under reduced pressure, and the residue was further purified by pre-HPLC under neutral conditions to give the product (41.45 mg, yield: 7.7%) as a solid. 1H NMR (DMSO-d6) δ 1.17-1.23 (m, 3 H), 1.68(s, 3 H), 3.56(s, 3 H), 3.65-3.82(m, 1 H), 4.13-4.35 (m, 4 H), 5.11-5.16(m, 1 H),6.25-6.29(m, 2 H), 7.16-7.21 (m, 1 H), 7.38-7.49 (m, 2 H), 7.63-7.64 (m, 1 H), 11.34 (d, 1 H); MS, m/e 538.4 (M+1)+.

### EXAMPLE 13

### Preparation of D-dioxolane-thymine 5′-(phenyl ethoxy-glycinyl phosphate)

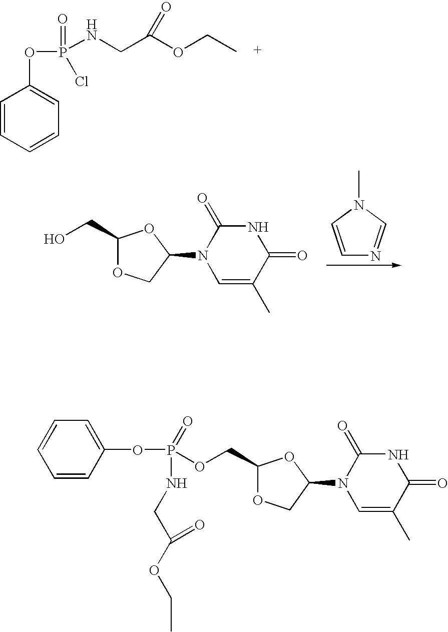

Phenyl ethoxy-glycinyl phosphorochloridate (1.02 g, 3.7 mmol) dissolved in 10 mL of anhydrous THF was added to a mixture of DOT (200 mg, 0.88 mmol) and N-methylimidazole (300 mg, 3.7 mmol) in 15 mL THF with vigorous stirring at room temperature, then the reaction was stirred overnight. Solvent was removed under reduced pressure, and the residue was further purified by pre-HPLC under neutral conditions to give the product (62.97 mg, yield: 15.26% as a solid). 1H NMR (DMSO-d6) δ 1.13-1.18 (m, 3 H), 1.71 (s, 3 H), 3.56-3.65 (m, 2 H), 4.03-4.15 (m, 3H), 4.26 (d, J=8.8 Hz, 3 H), 5.14(s, 1 H), 5.94-6.01 (m, 1H), 6.27-6.29 (m, 1 H), 7.16-7.19 (m, 3 H), 7.32-7.38 (m, 2 H), 7.44 (d, J=4.8 Hz, 1 H), 11.34 (s, 1H); MS, m/e 470.1 (M+1)+.

### EXAMPLE 14

### Preparation of D-dioxolane-thymine 5′-(phenyl benzyloxy-2-aminoisobutyric phosphate)

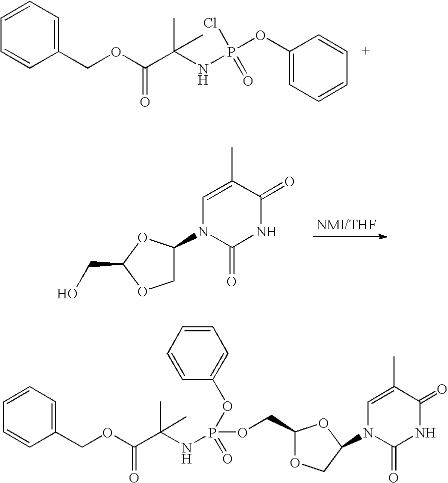

To the solution of DOT (200 mg, 0.9 mmol) and NMI (300 mg, 3.7 mmol) was dissolved in anhydrous THF (20 ml) was added dropewise phenyl benzyloxy-2-aminoisobutyric phosphorochloridate in THF (15 mL) at 0° C., then warm to room temperature and stirred overnight. The solvent was evaporated to dryness and purified by HPLC to give the product. (37.22 mg, Yield: 9.01%). ¹HNMR (400 MHz, DMSO): δ=1.28-1.33 (m, 3H), 1.35-1.39 (m, 3H), 1.67 (s, 3H), 4.09-4.26 (m, 4H), 5.06-5.09 (m, 3H), 5.99-6.02 (m, 1H), 6.27 (s, 1H), 7.13-7.17 (m, 3H), 7.28-7.35 (m, 7H), 7.37-7.41 (m, 1H), 11.32-11.34 (m, 1H). MS, m/e 559.95(M+1)⁺.

### EXAMPLE 15

### Preparation of D-dioxolane-thymine 5′-(phenyl benzyloxy-glycinyl phosphate)

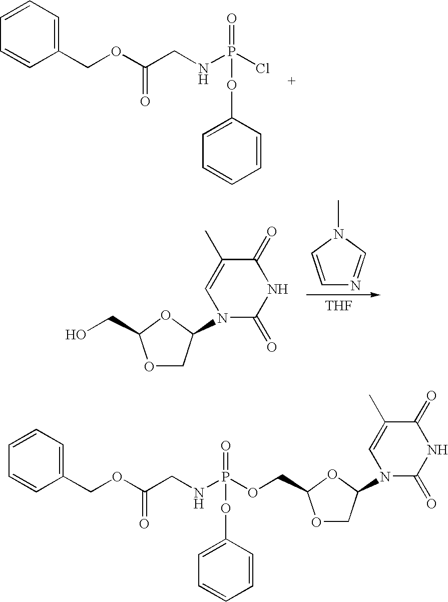

DOT (0.22 g, 1 eq.) and N-methylimidazole (0.61 g, 7.78 eq) were placed into a dry round bottl under nitrogen atmosphere. Using a dry syringe, anhydrous THF (40 ml) was added and the contents were stirred for additional 20 min. After this period, a solution of phenyl benzyloxy-glycinyl phosphorochloridate (1.41 g, 4.32 eq) in anhydrous THF (20 ml) was added and the mixture was stirred vigorously at room temperature over night. Then the solvent was removed under reduced pressure, and the residue was further purified by pre-HPLC under neutral condition to give the product as a white solid. (88.40 mg, 17.3%); ¹H NMR (DMSO-d₆) δ 1.70-1.70 (t, 3H), 3.65-3.75(m,2H),4.08-4.12 (m,1H), 4.24-4.27 (m, 3H), 5.10-5.13 (m, 3H), 6.01-6.10 (m, 1H), 6.28-6.30 (m, 1H), 7.16-7.18 (m,3H), 7.32-7.42 (m, 7H), 7.43-7.45 (m, 1H), 11.35 (s, 1H); MS, m/e 532.1 (M+1)⁻;

### EXAMPLE 16

### Preparation of D-dioxolane-thymine 5′-(phenyl methoxy-prolinyl phosphate)

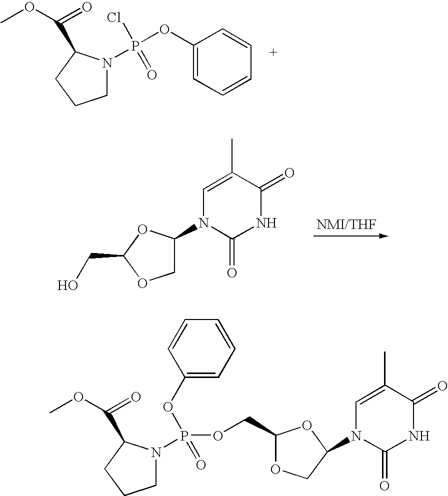

To the solution of DOT (200 mg, 0.9 mmol) and NMI (300 mg, 3.7 mmol) was dissolved in anhydrous THF (20 mL) was added phenyl methoxy-prolinyl phosphorochloridate in THF (10 mL), which was extracted with ether, stirred at room temperature for overnight. Then it was evaporated to dryness and purified by HPLC to give the product. (35.17 mg, Yield: 7.38%). ¹HNMR (400 MHz, DMSO): δ 1.65 (s, 3H), 1.75-1.81 (m, 2H), 1.96-2.00 (m, 1H), 3.06 (m, 1H), 3.16-3.20 (m, 1H), 3.47-3.55 (m, 3H), 4.04-4.10 (m, 2H), 4.14-4.26 (m, 2H), 4.34 (m, 1H), 5.06-5.12 (m, 1H), 6.22-6.24 (s, 1H), 7.08-7.15 (m, 3H), 7.26-7.34 (s, 2H), 7.39 (s, 1H), 11.30 (s, 1H). MS, m/e 495.93 (M+1)⁺.

### EXAMPLE 17 TO 121

Preparation of example compounds 17 to 121 were performed from the general procedures for DOT phosphoramidate derivatives as Example 3. The results are shown as the following table:

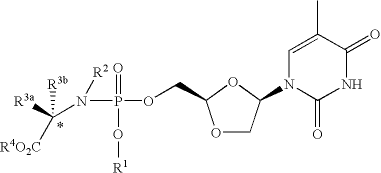

| Exs. | R¹ | R² | R^{3a} | R^{3b} | R⁴ | NMR and MS |
|---|---|---|---|---|---|---|
| 17 | Ph | H | H | Me | pentyl | δ 0.80 (d, J = 4 Hz, 3H), 1.14-1.22 (m, 7H), 1.46-1.49 (m, 2H), 1.68 (d, J = 4.4 Hz, 3H), 3.70-3.86 (m,1H), 3.93-4.22 (m, 6H), 5.10 (d, J = 16 Hz, 1H), 5.98-6.09 (m, 1H), 6.25 (s, 1H), 7.11-7.14 (m, 3H), 7.29-7.42 (m, 2H), 7.40 (d, J = 14 Hz, 1H), 11.51 (s, 1H);MS, m/e 525.9 (M + 1)⁺; 1072.74 (2M + 23)⁺ |
| 18 | Ph | H | H | Me | hexyl | δ 0.74 (m, 3H), 1.12 (m, 9H), 1.44 (m, 2H), 1.64(d, J = 4.8 Hz, 3H), 3.70 (m, 1H), 3.89 (m, 2H), 4.15(m, 4H), 5.10 (s, 1H), 6.00 (m, 2H), 6.21 (t,J = 4.4 Hz, 1H), 7.09 (m, 3H), 7.27 (m, 3H),11.35 (s, 1H); MS, m/e 539.9 (M + 1)⁺; |
| 19 | Ph | H | H | Me | 4-F-Bn | δ 1.18-1.25 (m, 3H), 1.69 (d, J = 8.0 Hz, 3H), 3.82-3.89 (m, 1H), 4.09-4.28 (m, 4H), 5.04-5.12 (m, 3H),6.07-6.18 (m, 1H), 6.28 (s, 1H), 7.11-7.20 (m, 5H),7.30-7.43 (m, 5H), 11.35 (s, 1H). MS, m/e 563.94(M + 1)⁺; |
| 20 | 4-Cl-Ph | H | H | Me | Et | δ 0.74 (d, J = 6.8 Hz, 3H), 1.12 (m, 9H), 1.44 (m,2H), 1.64 (d, J = 4.8 Hz, 3H), 3.70 (m, 1H), 3.89 (m,2H), 4.15 (m, 4H), 5.10 (s, 1H), 6.00 (m, 2H), 6.21(t, J = 4.4 Hz, 1H), 7.09 (m, 3H), 7.27 (m, 3H),11.35 (s, 1H); MS, m/e 517.78 (M + 1)⁺; |
| 21 | 4-Cl-Ph | H | H | Me | i-Pr | δ 1.09-1.19 (m, 9H), 1.68 (d, J = 4.8 Hz, 3H), 4.05-4.23 (m, 4H), 4.80-4.81 (m, 1H), 5.11 (d, J = 9.2 Hz1H), 6.05-6.12 (m, 1H), 6.25 (d, 1H), 7.13-7.18 (m,2H), 7.36-7.40 (m, 1H), 11.31 (d, 1H); MS, m/e531.9 (M + 1)⁺; 1084.71 (2M + 23)⁺ |
| 22 | 4-Cl-Ph | H | H | Me | n-Bu | δ 0.82 (m, 3H), 1.25 (m, 6H), 1.50 (m, 2H), 1.70(m, 3H) 3.70 (m, 1H), 3.95 (m, 2H), 4.17-4.3 (m,4H), 5.10 (s, 1H), 6.10 (m, 1H), 6.35 (m, 1H), 7.18(m, 2H), 7.40 (m, 3H), 11.4 (s, 1H); MS, m/e 546(M + 1)⁺; |
| 23 | 2-Cl-Ph | H | H | Me | i-Pr | δ: 1.11-1.16 (m, 6H), 1.21-1.23 (m, 3H), 1.67 (s,3H), 3.72-3.85 (m, 1H), 4.14 (t, J = 12.0 Hz, 1H),4.26-4.28 (m, 3H), 4.82-4.84 (m, 1H), 5.16 (d,J = 12.0 Hz, 1H), 6.27-6.28 (m, 2H), 7.18 (t,J = 8.0 Hz, 1H), 7.31 (m, 1H), 7.41-7.51 (m, 3H),11.37 (s, 1H): MS, m/e 531.88 (M + 1)⁺; |
| 24 | 2-Cl-Ph | H | H | Me | n-Bu | δ: 0.82-0.88 (m, 3H), 1.24-1.30 (m, 5H), 1.47-1.51(m, 2H), 1.68-1.69 (s, 3H), 3.76-3.80 (m, 1H), 3.97-4.02 (m, 2H), 4.14 (m, 1H), 4.26-4.29 (m, 3H), 5.17(d, J = 12.0 Hz, 1H), 6.27-6.30 (m, 2H), 7.18 (t,J = 8.0 Hz, 1H), 7.32-7.33 (m, 1H), 7.41 (m, 1H),7.44-7.47 (m, 1H), 7.51 (d, J = 8.0 Hz, 1H),11.37 (s, 1H); MS, m/e 545.89 (M + 1)⁺; |
| 25 | 2-Cl-Ph | H | H | Me | Bn | δ 1.12 (m, 3H), 1.78 (d, J = 13.2 Hz, 3H), 3.70 (m,1H), 4.11 (s, 1H), 4.26 (t, J = 2.4 Hz, 3H), 5.09 (m,3H), 6.27 (d, J = 22.4 Hz, 2H), 7.10-7.50 (m, 10H),11.4 (s, 1H); MS, m/e 579.87 (M + 1)⁺/ 596.78(M + 18)⁺; |
| 26 | 4-Br-Ph | H | H | Me | Et | δ: 1.11-1.20 (m, 7H), 1.71 (d, J = 8.0 Hz, 3H), 4.01-4.02 (m, 2H), 4.03-4.04 (m, 1H), 4.26 (m, 3H), 5.14(d, J = 8.0 Hz, 1H), 6.08-6.19 (m, 1H), 6.28-6.31 (m,1H), 7.11-7.14 (m, 2H), 7.43 (d, J = 8.0 Hz, 1H),7.54-7.56 (m, 2H); MS, m/e 561.80 (M + 1)⁺; |
| 27 | 4-Br-Ph | H | H | Me | i-Pr | δ: 1.12-1.22 (m, 9H), 1.70-1.72 (s, 3H), 3.69-3.71(m, 1H), 4.11-4.15 (m, 1H), 4.21-4.29 (m, 3H), 4.81-4.84 (m, 1H), 5.15 (d, J = 8.0 Hz, 1H), 6.07-6.13 (m,1H), 6.29 (d, J = 8.0 Hz, 2H), 7.11-7.16 (m, 2H),7.42 (d, J = 12.0 Hz, 1H), 7.52-7.56 (m, 2H), 11.35 (s,1H); MS, m/e 575.80 (M + 1)⁺; |
| 28 | 4-Br-Ph | H | H | Me | n-Bu | δ: 0.85-0.93 (m, 3H), 1.27-1.45 (m, 5H), 1.47-1.51(m, 2H), 1.72 (d, J = 8.0 Hz, 3H), 3.72-3.84 (m, 1H),3.97-4.01 (m, 2H), 4.13-4.16 (m, 1H), 4.13-4.29 (m,3H), 5.16 (d, J = 4.0 Hz, 1H), 6.12-6.19 (m, 1H), 6.29(d, J = 8.0 Hz, 1H), 7.12-7.14 (m, 2H), 7.44 (d, J =4.0 Hz, 1H), 7.53-7.56 (m, 2H), 11.31 (s, 1H); MS,m/e 591.88 (M + 3)⁺; |
| 29 | 4-Br-Ph | H | H | Me | hexyl | δ 0.83-084 (m, 3H), 1.17-1.26 (m, 9H), 1.46-1.49 (m,2H), 1.70 (d, J = 5.6 Hz, 3H), 3.71-3.84 (m, 1H), 3.98-3.99 (m, 2H), 4.10-4.27 (m, 4H), 5.12 (d, J = 14.4 Hz,1H), 6.12-6.13 (m, 1H), 6.26-6.28 (m, 1H), 7.12 (t,J = 7.6 Hz, 2H), 7.40 (dd, J = 11.2, 1.2 Hz, 1H), 7.53(t, J = 4.4 Hz 2H), 11.32 (s, 1H) ; LCMS, m/e 618.1(M + 1)⁺; |
| 30 | 4-Br-Ph | H | H | Me | propyl | δ 0.80-0.87 (m, 3H), 1.20 (dd, J = 14.8, 6.8 Hz 3H),1.51-1.54 (m, 2H), 1.69-1.71 (m, 3H), 3.72-3.86 (m,1H), 3.92-3.95 (m, 2H), 4.13-4.27 (m, 4H), 5.14 (dd,J = 16, 1.6 Hz 1H), 6.10-6.15 (m, 1H), 6.27-6.29 (m,1H), 7.10-7.14 (m, 2H), 7.39 (dd, J = 12.4, 1.2 Hz1H), 7.51-7.55 (m, 2H), 11.32 (d, J = 5.6 Hz 1H)MS, m/e 575.82 (M + 1)⁺; 1174.54 (2M + 23)⁺ |
| 31 | 4-Br-Ph | H | H | Me | pentyl | δ 0.82-0.86 (m, 3H), 1.14-1.23 (m, 7H), 1.45-1.48(m, 2H), 1.68 (dd, J = 6, 0.8 Hz, 3H), 3.69-3.81 (m,1H), 3.91-3.98 (m, 2H), 4.08-4.26 (m, 4H), 5.10 (dd,J = 14.4, 0.8 Hz, 1H), 6.10-6.11 (m, 1H), 6.24-6.27(m, 1H), 7.08-7.12 (m, 2H), 7..37-7.40 (m, 1H),7.49-7.53 (m, 2H), 11.31 (d, J = 5.2 Hz, 1H);LCMS, m/e 606.0 (M + 1)⁺; |
| 32 | 4-Br-Ph | H | H | Me | 2-Bu | δ: 0.86 (d, J = 2.4 Hz, 6H), 1.21 (dd, J = 6.4 Hz, 0.8 Hz,3H), 1.72 (dd, J = 7.6 Hz, 1.2 Hz, 3H), 1.80-1.84 (m,1H), 3.75-3.84 (m, 3H), 4.14-4.16 (m, 1H), 4.24-4.27 (m, 3H), 5.14 (d, J = 13.6 Hz, 1H), 6.12-6.20 (m,1H), 6.28-6.30 (m, 1H), 7.12-7.16 (m, 2H), 7.44 (d,J = 1.2 Hz, 1H), 7.54 (dd, J = 8.4 Hz, 1.2 Hz, 2H),11.35 (s, 1H); MS, m/e 589.87 (M + 1)⁺; |
| 33 | 4-Br-Ph | H | H | Me | cyclo-hex | δ: 1.18-1.30 (m, 9H), 1.62-1.72 (m, 7H), 3.70-3.85(m, 1H), 4.11-4.29 (m, 4H), 4.60 (s, 1H), 5.14 (d, J =14.4 Hz, 1H), 6.11-6.15 (m, 1H), 6.27-6.30 (m, 1H),7.11-7.15 (m, 2H), 7.40-7.43 (m, 1H), 7.52-7.56 (m,2H), 11.35 (s, 1H); MS, m/e 615.85 (M + 1)⁺ |
| 34 | 4-Br-Ph | H | H | Me | t-Bu | δ: 1.15-1.20 (m, 3H), 1.34-1.39 (m, 9H), 1.71 (d, J =4.4 Hz, 3H), 3.60-3.68 (m, 1H), 4.11-4.29 (m, 4H),5.14 (d, J = 14.0 Hz, 1H), 6.03 (t, J = 10.4 Hz, 1H),6.28 (t, J = 10.0 Hz, 1H), 7.11-7.17 (m, 2H), 7.42 (d,J = 10.0 Hz, 1H), 7.51-7.59 (m, 2H), 11.35 (s, 1H);MS, m/e 589.68 (M + 1)⁺, 591.66 (M + 3)⁺ |
| 35 | 4-F-Ph | H | H | Me | Et | δ: 1.12-1.35 (m, 6H), 1.70-1.72 (d, 3H), 3.70-3.85 (m, 1H), 4.00-4.30 (m, 6H), 5.12-5.16 (d, 1H),6.04-6.11 (m, 1H), 6.28-6.30 (m, 1H), 7.18-7.20(m, 4H), 7.41-7.44 (d, 2H), 11.35 (s.1H); MS, m/e502.1 (M + 1)⁺; |
| 36 | 4-F-Ph | H | H | Me | i-Pr | δ: 1.13-1.23 (m, 9H), 1.70-1.72 (d, 3H), 3.65-3.80 (m, 1H), 4.11-4.30 (m, 5H), 4.82-4.86 (m, 1H),5.12-5.16 (d, 1H), 6.04-6.10 (m, 1H), 6.28-6.30 (m,1H), 7.18-7.20 (m, 4H), 7.41-7.44 (d, 2H), 11.35(d.1H); MS, m/e 515.90 (M + 1)⁺; |
| 37 | 4-F-Ph | H | H | Me | n-Bu | δ: 0.82-0.86 (m, 3H), 1.17-1.30 (M, 5H), 1.46-1.52(m, 2H), 1.70-1.72 (d, 3H), 3.70-3.85 (m, 1H) , 4.00-4.30 (m, 6H), 5.12-5.16 (d, 1H), 6.04-6.11 (m, 1H),6.28-6.30 (m, 1H), 7.18-7.20 (m, 4H), 7.41-7.44 (d,2H), 11.35 (d.1H): MS, m/e 530.1 (M + 1)⁺; |
| 38 | 4-F-Ph | H | H | Me | Bn | δ: 1.20-1.35 (m, 3H), 1.68-1.70 (d, 3H), 3.83-3.89(m, 1H) , 4.10-4.30 (m, 4H), 5.08-5.12 (m, 3H), 6.11-6.20 (m, 1H), 6.27-6.29 (m, 1H), 7.14-7.20 (m, 4H),7.34-7.44 (m, 6H), 11.35 (d.1H); MS, m/e 563.87(M + 1)⁺; |
| 39 | 2,4-di-Cl-Ph | H | H | Me | Et | δ: 1.10 (m, 3H), 1.20 (m, 3H), 1.70 (d, J = 4.8, 3H),3.80 (m, 1H), 4.05 (m, 2H), 4.17 (m, 1H), 4.38 (m,3H), 5.17 (s, 1H), 6.32 (m, 2H), 7.43 (m, 3H),7.70 (s, 1H), 11.4 (s, 1H); MS, m/e 551.78 (M + 1)⁺/568.7 (M + 18)⁺; |
| 40 | 2,4-di-Cl-Ph | H | H | Me | i-Pr | δ: 1.10 (m, 6H), 1.19 (t, J = 6.8 Hz, 3H), 1.65 (s,3H), 3.75 (m, 1H), 4.11 (d, J = 6 Hz, 1H), 4.26 (m,3H), 4.79 (d, J = 6.4 Hz, 1H), 5.13 (s, 1H), 6.25 (d,J = 4 Hz, 2H), 7.38 (m, 3H), 7.67 (s, 1H), 11.4 (s,1H); MS, m/e 565.8 (M + 1)⁺; |
| 41 | 2,4-di-Cl-Ph | H | H | Me | n-Bu | δ: 0.85 (m, 3H), 1.25 (m, 5H), 1.50 (m, 2H), 1.70(s, 3H), 3.80 (m, 1H), 3.92 (m, 2H), 4.05 (m, 1H),4.26 (m, 3H), 5.10 (s, 1H), 6.26 (m, 2H), 7.43 (m,3H), 7.67 (s, 1H), 11.4 (s, 1H); MS, m/e 579.78(M + 1)⁺/596.7 (M + 18)⁺; |
| 42 | 2,4-di-Cl-Ph | H | H | Me | Bn | δ: 1.26 (d, J = 7.2 Hz, 3H), 1.66 (s, 3H), 3.90 (m,1H), 4.12 (s, 1H), 4.26 (m, 3H), 5.10 (m, 3H),6.26 (m, 1H), 6.41 (m, 1H), 7.36 (m, 8H), 7.67 (d,J = 3.2 Hz, 1H), 11.35 (s, 1H); MS, m/e 613.9(M + 1)⁺; |
| 43 | 3,4-di-Cl-Ph | H | H | Me | Et | δ: 1.11-1.26 (m, 6H), 1.70-1.72 (d, 3H), 3.75-3.85 (m, 1H), 4.00-4.10 (m, 2H), 4.12-4.18 (m, 1H),4.22-4.34 (m, 3H), 5.15-5.18 (d, 1H), 6.23-6.31 (m,2H), 7.17-7.20 (m, 1H), 7.40-7.50 (m, 2H), 7.62-7.67 (t, 1H), 11.35 (d.1H); MS, m/e 551.81 (M + 1)⁺; |
| 44 | 3,4-di-Cl-Ph | H | H | Me | i-Pr | δ: 1.10 (m, 9H), 1.66 (s, 3H), 3.67 (d, J = 7.2 Hz,1H), 4.12 (m, 1H), 4.20 (m, 3H), 4.79 (t, J = 6.4 Hz,1H), 5.13 (s, 1H), 6.24 (m, 2H), 7.13 (d, J = 9.2 Hz,1H), 7.36 (t, J = 14 Hz, 2H), 7.59 (d, J = 8.8 Hz, 1H),11.35 (s, 1H); MS, m/e 565.9 (M + 1)⁺; |
| 45 | 3,4-di-Cl-Ph | H | H | Me | n-Bu | δ: 0.82-0.88 (m, 3H), 1.20-1.30 (m, 5H), 1.45-1.52(m, 2H), 1.69-1.71 (d, 3H), 3.75-3.85 (m, 1H) , 3.96-4.05 (m, 2H), 4.12-4.18 (m, 1H), 4.26-4.34 (m, 3H),5.15-5.17 (d, 1H), 6.27-6.31 (m, 2H), 7.17-7.20(m, 1H), 7.40-7.50 (m, 2H), 7.63-7.67 (t, 1H), 11.35(d.1H) ; MS, m/e 579.83 (M + 1)⁺; |
| 46 | 3,4-di-Cl-Ph | H | H | Me | Bn | δ: 1.21 (m, 3H), 1.66 (d, J = 4.8 Hz, 3H), 3.85 (t,J = 8.6 Hz, 1H), 4.12 (m, 1H), 4.24 (d, J = 9.6 Hz,3H), 5.08 (m, 3H), 6.32 (m, 2H), 7.14 (t, J = 3.6 Hz,2H), 7.41 (m, 5H), 7.45 (m, 2H), 7.58 (d, J = 8.8 Hz,1H), 11.35 (s, 1H); MS, m/e 614.1/616.4 (M + 1)⁺; |
| 47 | 4-MeO-Ph | H | H | Me | i-Pr | δ: 1.10-1.23 (m, 9H), 1.72 (d, J = 4.0 Hz, 3H), 3.67-3.75 (m, 4H), 4.09-4.29 (m, 4H), 4.83-4.87 (m, 1H),5.12 (d, J = 8.0 Hz, 1H), 5.91-6.01 (m, 1H), 6.27-6.30(m, 1H), 6.88 (dd, J = 8.0 4.0 Hz, 2H), 7.08 (dd,J = 12.0 2.0 Hz, 2H), 7.44 (d, J = 16.0 Hz, 1H), 11.33 (s,1H); MS, m/e 527.91 (M + 1)⁺; |
| 48 | 4-MeO-Ph | H | H | Me | n-Bu | δ: 0.80-0.91 (m, 3H), 1.13-1.28 (m, 5H), 1.47-1.53(m, 2H), 1.72 (d, J = 8.0 Hz, 3H), 3.72-3.77 (m, 4H),3.99-4.03 (m, 2H), 4.12-4.29 (m, 4H), 5.13 (d, J =16.0 Hz, 1H), 5.96-6.00 (m, 1H), 6.29 (dd, J = 12.08.0 Hz, 1H), 6.88 (dd, J = 8.0 4.0 Hz, 2H), 7.07-7.10(m, 2H), 7.44 (d, J = 12.0 Hz, 1H), 11.35-11.39 (s,1H); MS, m/e 541.95 (M + 1)⁺; |
| 49 | 4-Me-Ph | H | H | Me | i-Pr | δ: 1.12-1.20 (m, 9H), 1.70 (d, J = 3.6 Hz, 3H), 2.25(s, 3H), 3.60-3.79 (m, 1H), 4.13-4.25 (m, 4H), 5.143(d, 1H), 5.96-6.05 (m, 1H), 6.27-6.29 (m, 1H), 7.01-7.14 (m, 4H), 7.42 (d, J = 16 Hz, 1H), 11.31 (d, 1H);MS, m/e 511.9 (M + 1)⁺; 1044.74 (2M + 23)⁺ |
| 50 | 4-Me-Ph | H | H | Me | n-Bu | δ: 0.78-0.89 (m, 3H), 1.14-1.27 (m, 5H), 1.45-1.50(m, 2H), 1.68 (d, J = 4.4 Hz, 1H), 2.22 (s, 3H), 3.70-3.80 (m, 1H), 3.96-3.98 (m, 2H), 4.08-4.25 (m,4H), 5.10 (d, J = 15.6 Hz, 1H), 5.90-6.05 (m, 1H),6.25-6.27 (m, 1H), 6.99-7.11 (m, 4H), 7.40 (d,J = 15.6 Hz, 1H), , 11.31 (s, 1H); MS, m/e525.98 (M + 1)⁺; 1072.78 (2M + 23)⁺ |
| 51 | 4-Me-Ph | H | H | Me | Bn | δ: 1.18-1.25 (m, 3H), 1.68 (d, J = 6 Hz, 3H), 2.24 (s,3H) 3.78-3.92 (m, 1H), 4.09-4.27 (m, 4H), 5.06-5.10(m, 3H), 6.02-6.15 (m, 1H), 6.26-6.28 (m, 1H), 6.98-7.03 (m, 2H), 7.08-7.11 (m, 2H), 7.33-7.43 (m,6H), 11.31 (s, 1H); MS, m/e 582.2 (M + 23)⁺; 1140.73(2M + 23)⁺ |
| 52 | Ph | H | H | i-Bu(Leu) | Me | δ .066-0.80 (m, 6H), 1.35-1.39 (m, 3H), 1.68-1.69(m, 3H), 3.54 (d, J =2.4 Hz 3H), 3.62-3.74 (m, 1H),4.11-4.26 (m, 4H), 5.14 (d, J = 1.6 Hz 1H), 5.99-6.11 (m, 1H), 6.26-6.28 (m, 1H), 7.10-7.15 (m, 3H),7.30-7.38 (m, 2H), 7.44 (d, J = 1.2 Hz 1H), 1.31 (s,1H); MS, m/e 511.96 (M + 1)⁺; 1044.73 (2M + 23)⁺ |
| 53 | Ph | H | H | 3-indolyl-CH₂-(Try) | Me | δ = 1.62 (d, J = 1.2 Hz, 3H), 2.91-2.93 (m, 1H), 3.04(s, 1H), 3.45 (m, 3H), 3.93-4.04 (m, 2H), 4.05-4.10(m, 2H), 4.19 (d, J = 8.0 Hz, 1H), 4.98 (s, 1H), 6.05-6.11 (m, 1H), 6.22 (dd, J = 6.0 Hz, 2.0 Hz, 1H), 6.90-6.95 (m, 3H), 6.97-7.00 (m, 3H), 7.21-7.25 (m, 2H),7.28-7.33 (m, 1H), 7.34-7.39 (m, 2H), 10.81 (s, 1H),11.28 (d, J = 18.0 Hz, 1H); MS, m/e 584.98 (M + 1)⁺; |
| 54 | Ph | H | H | Sec-Butyl(Ile) | Me | δ .067-0.77 (m, 6H), 0.99-1.33 (m, 1H), 1.28-1.41 (m, 1H), 1.57-1.71 (m, 4H), 3.51-3.55 (m, 4H),4.10-4.24 (m, 4H), 5.13 (d, J = 1.6 Hz 1H), 5.91-6.11(m, 1H), 6.26-6.28 (m, 1H), 7.13-7.16 (m, 3H), 7.31-7.40 (m, 2H), 7.43 (d, J = 1.2 Hz 1H), 1.31 (s, 1H);MS, m/e 511.94 (M + 1)⁺; 1044.74 (2M + 23)⁺ |
| 55 | Ph | H | H | Methyl-mercapto-Et(Met) | Me | δ 1.71 (d, J = 10.8 Hz, 3H), 1.76-1.80 (m, 2H), 1.92(d, J = 14.0 Hz, 3H), 2.22-2.27 (m, 1H), 2.37-2.46(m, 1H), 3.55 (s, 3H), 3.79-3.82 (m, 1H), 4.09-4.12(m, 1H), 4.20-4.25 (m, 3H), 5.11 (d, J = 20.0 Hz,1H), 6.04-6.18 (m, 1H), 6.24-6.26 (m, 1H), 7.11-7.15 (m, 3H), 7.28-7.37 (m, 2H), 7.42 (s, 1H), 11.30(s, 1H); MS, m/e 529.92 (M + 1)⁺; |
| 56 | 4-Br-Ph | H | H | i-Butyl(Leu) | Me | δ: 0.65-0.75 (m, 3H), 0.75-0.84 (m, 3H), 1.36-1.65(m, 3H), 1.71 (d, J = 6.0 Hz, 3H), 3.57 (d, J = 6.8 Hz,3H), 3.66-3.76 (m, 1H), 4.11-4.19 (m, 2H), 4.20-4.32 (m, 2H), 5.12-5.16 (m, 1H), 6.06-6.18 (m, 1H),6.27-6.30 (m, 1H), 7.12 (t, J = 8.2 Hz, 2H), 7.42 (d,J = 21.2 Hz, 1H), 7.54 (t, J = 8.6 Hz, 2H), 11.35 (s,1H); MS, m/e 589.78 (M + 1)⁺; |
| 57 | 4-Br-Ph | H | H | i-Bu (Leu) | Et | δ: 0.67-0.77 (m, 3H ), 0.82 (dd, J = 14.0, 6.4 Hz,3H), 1.13-1.16 (m, 3H), 1.38-1.46 (m, 2H), 1.45-1.63 (m, 1H), 1.60-1.65 (m, 3H), 3.64-3.71 (m, 1H),4.00-4.10 (m, 2H), 4.11-4.18 (m, 2H), 4.20-4.29 (m,2H), 5.12-5.16 (m, 1H), 6.04-6.18 (m, 1H), 6.28-6.29 (m, 1H), 7.12 (t, J = 8.0 Hz, 2H), 7.43 (d, J =20.8 Hz, 1H), 7.51-7.54 (m, 2H), 11.34 (s, 1H); MS,m/e 603.91 (M + 1)⁺ |
| 58 | 4-Br-Ph | H | H | i-Bu (Leu) | i-Pr | δ: 0.67-0.83 (m, 6H), 1.11-1.14 (m, 6H), 1.36-1.44 (m, 3H), 2.49 (s, 3H), 3.58-3.63 (m, 1H) 4.11-4.27 (m, 4H), 4.81-4.86 (m, 1H), 5.13 (d, J = 16.4 Hz1H), 5.99-6.05 (m, 1H), 6.28 (d, J = 4.8 Hz 1H),7.09-7.13 (m, 2H), 7.42 (d, J = 20 Hz 1H), , 7.50-7.55 (m, 2H), 11.32 (d, J = 7.6 Hz 1H); MS, m/e617.85 (M + 1)⁺; |
| 59 | 4-Br-Ph | H | H | i-Bu (Leu) | n-Bu | δ: 0.68-0.86 (m, 9H), 1.24-1.51 (m, 7H), 1.71-1.72(m, 3H), 3.61-3.72 (m, 1H), 3.96-3.99 (m, 2H), 4.14-4.27 (m, 4H), 5.15 (d, J = 17.6 Hz, 1H), 6.02-6.16(m, 1H), 6.28 (d, J = 5.2 Hz 1H), 7.09-7.13 (m,2H), 7.41 (d, J = 18.8 Hz 1H) 7.50-7.55 (m,2H), 11.32 (d, J = 6.4 Hz 1H); MS, m/e 631.93(M + 1)⁺; |
| 60 | 4-Br-Ph | H | H | i-Bu (Leu) | Bn | δ 0.67-0.77 (m, 3H), 0.82 (dd, J = 14.0, 6.4 Hz, 3H),1.40-1.45 (m, 2H), 1.45-1.52 (m, 1H), 1.72 (s, 3H),3.70-3.79 (m, 1H), 4.09-4.28 (m, 4H), 5.07-5.11 (m,3H), 6.09-6.25 (m, 1H), 6.28 (d, J = 4.2 Hz, 1H),7.07-7.12 (m, 2H), 7.32-7.43 (m, 5H), 7.42 (d, J =18.0 Hz, 1H), 7.46-7.54 (m, 2H), 11.33 (s, 1H); MS,m/e 665.91 (M + 1)⁺ |
| 61 | 4-Br-Ph | H | Me | H | Me | δ: 1.18-1.21 (m, 3H), 1.71 (s, 3H), 3.56-3.58 (m,3H), 3.78-3.81 (m, 1H), 4.11-4.15 (m, 2H), 4.24-4.28 (m, 2H), 5.13 (d, J = 14.4 Hz, 1H), 6.18-6.21(m, 1H), 6.27-6.30 (m, 1H), 7.10-7.17 (m, 2H), 7.41-7.43 (m, 1H), 7.43-7.57 (m, 2H), 11.35 (s, 1H); Ms:m/e 549.84 (M + 2)⁺, 570.00 (M + 23)⁺ |
| 62 | 4-Br-Ph | H | Me | H | n-Bu | δ: 0.82-0.87 (m, 3H), 1.19-1.28 (m, 5H), 1.46-1.50(m, 2H), 1.72 (s, 3H), 3.72-3.85 (m, 1H), 3.96-4.00(m, 2H), 4.13-4.26 (m, 4H), 5.14 (d, J = 13.2 Hz,1H), 6.10-6.20 (m, 1H), 6.28 (s, 1H), 7.14 (dd, J =18.4 Hz, 8.0 Hz, 2H), 7.43 (d, J = 6.0 Hz, 1H), 7.53-7.56 (m, 2H), 11.35 (s, 1H); Ms: m/e 589.79 (M + 1)⁺ |
| 63 | 4-Br-Ph | H | Me | H | Bn | δ: 1.12-1.25 (m, 3H), 1.69-1.70 (m, 3H), 3.81-3.90 (m, 1H), 4.10-4.27 (m, 4H), 5.02-5.12 (m, 3H),6.19-6.22 (m, 1H), 6.28 (d, J = 5.6 Hz 1H), 7.11 (dd,J = 22.4, 8.8 Hz 2H), 7.15-7.41 (m, 6H), 7.51 (d , J =8.4 Hz 2H), 11.33 (d, J = 6 Hz 1H); MS, m/e623.87 (M + 1)⁺; |
| 64 | 4-F-Ph | H | H | i-Bu (Leu) | Me | δ: 0.67-0.77 (m, 3H), 0.82 (dd, J = 14.0, 6.4 Hz,3H), 1.49-1.72 (m, 3H), 1.72 (d, J = 3.2 Hz, 3H),3.58 (d, J = 7.6 Hz, 3H), 3.65-3.75 (m, 1H), 4.10-4.18 (m, 2H), 4.19-4.30 (m, 2H), 5.12-5.16 (m, 1H),6.00-6.15 (m, 1H), 6.28-6.29 (m, 1H), 7.14-7.23 (m,4H), 7.43 (d, J = 23.2 Hz, 1H), 11.33 (s, 1H); MS,m/e 529.90 (M + 1)⁺ |
| 65 | 4-F-Ph | H | H | i-Bu (Leu) | Bn | δ: 0.64-0.82 (m, 6H), 1.39-1.47 (m, 3H), 1.69-1.70(m, 3H), 3.68-3.76 (m, 1H)□ 4.11-4.26 (m, 4H), 5.07-5.10 (m, 3H), 6.03-6.17 (m, 1H), 6.26-6.28 (m, 1H),7.12-7.17 (m, 4H), 7.31-7.35 (m, 5H), 7.41 (dd, J =20.4, 1.2 Hz 1H), 11.32 (d, J = 8.4 Hz 1H); MS, m/e605.99 (M + 1)⁺; |
| 66 | 4-F-Ph | H | Me | H | Me | δ 1.20 (t, J = 15.6 Hz, 3H), 1.72 (d, J = 3.2 Hz, 3H),3.58 (d, J = 7.2 Hz, 3H), 3.70-3.85 (m, 1H), 4.12-4.28 (m, 4H), 5.14 (d, J = 14.4 Hz, 1H), 6.10-6.13(m, 1H), 6.27-6.29 (m, 1H), 7.17-7.22 (m, 4H), 7.42-7.44 (m, 1H), 11.35 (s, 1H); Ms: m/e 487.85 (M + 1)⁺; |
| 67 | 4-F-Ph | H | Me | H | Bn | δ 1.24 (dd, J = 10.4 Hz, 6.8 Hz, 3H), 1.69-1.72 (m,3H), 3.80-3.95 (m, 1H), 4.13-4.26 (m, 4H), 5.06-5.12 (m, 3H), 6.10-6.22 (m, 1H), 6.29 (d, J = 4.0 Hz,1H), 7.14-7.20 (m, 4H), 7.32-7.36 (m, 5H), 7.43 (s,1H), 11.35 (s, 1H); Ms: m/e 586.05 (M + 23)⁺ |
| 68 | 4-Cl-Ph | H | H | i-Bu (Leu) | Me | δ 0.64-0.81 (m, 6H), 1.35-1.42 (m, 3H), 1.68-1.69 (m, 3H), 3.54-3.57 (m, 3H), 3.66-3.69 (m, 1H),4.08-4.25 (m, 4H), 5.11 (d, J = 17.6 Hz, 1H), 6.02-6.06 (m, 1H), 6.25-6.26 (m, 1H), 7.12-7.16 (m, 2H),7.36-7.42 (m, 3H), 11.31 (d, J = 7.6 Hz 1H); MS,m/e 545.91 (M + 1)⁺; |
| 69 | 4-Cl-Ph | H | H | i-Bu (Leu) | Bn | δ 0.62-0.79 (m, 6H), 1.32-1.61 (m, 3H), 1.66-1.67(m, 3H), 3.67-3.75 (m, 1H), 4.08-4.21 (m, 4H), 5.03-5.08 (m, 3H) 6.02-6.20 (m, 1H), 6.24 (d, J = 5.6 Hz1H), 7.11-7.13 (m, 2H), 7.29-7.40 (m, 8H), 11.31 (s,1H); MS, m/e 631.93 (M + 1)⁺; |
| 70 | 4-Cl-Ph | H | Me | H | Me | δ 1.21 (m, J = 6.8 Hz, 3H), 1.72 (s, 3H), 3.58 (m,3H), 3.75-3.83 (m, 1H), 4.11-4.34 (m, 4H), 5.14 (d,J = 14.8 Hz, 1H), 6.12-6.20 (m, 1H), 6.29 (s, 1H),7.14-7.23 (m, 2H), 7.34-7.44 (m, 3H), 11.35 (s, 1H);Ms: m/e 503.85 (M + 1)⁺, 526.08 (M + 23)⁺ |
| 71 | 4-Cl-Ph | H | Me | H | Bn | δ: 1.20-1.27 (m, 3H), 1.70 (d, J = 7.6 Hz, 3H), 3.86-3.90 (m, 1H), 4.10-4.28 (m, 4H), 5.06-5.12 (m, 3H),6.16-6.29 (m, 2H), 7.13-7.21 (m, 2H), 7.31-7.42 (m,8H), 11.35 (s, 1H); Ms: m/e 579.84 (M + 1)⁺ |
| 72 | Ph | H | H | Me | Cyc-hex | δ: 1.17-1.22 (m, 4H), 1.23-1.35 (m, 4H), 1.44-1.46(m, 1H), 1.63-1.73 (m, 7H), 3.70-3.82 (m, 1H), 4.10-4.29 (m, 4H), 4.59-4.66 (m, 1H)□5.12-5.16 (m, 1H),6.01-6.11 (m, 1H), 6.28-6.31 (m, 1H), 7.15-7.18 (m,3H), 7.32-7.37 (m, 2H), 7.42-7.46 (m, 1H), 11.33(d, J = 10.0 Hz, , 1H); MS, m/e 538.01 (M + 1)⁺; |
| 73 | Ph | H | H | Me | Cyc-pent | δ 1.14-1.20 (m, 3H), 1.51-1.1.59 (m, 6H), 1.70-1.77(m, 5H), 3.66-3.78 (m, 1H), 4.09-4.28 (m, 4H), 5.00(s, 1H), 5.13 (d, J = 14.8 Hz 1H), 5.99-6.09 (m, 1H),5.13 (d, J = 14.8 Hz 1H), 6.28 (d, J = 4.4 Hz 1H), 7.14-7.17 (m, 3H), 7.31-7.37 (m, 2H), 7.43 (d, J = 15.6 Hz1H), 11.35 (s, 1H); MS, m/e 523.98 (M + 1)⁺; |
| 74 | 4-Br-Ph | H | H | Me | Cyc-pent | δ 1.18 (dd, J = 8.0 Hz, 8.4 Hz, 3H), 1.50-1.58 (m,6H), 1.70-1.72 (m, 5H), 3.62-3.75 (m, 1H), 4.10-4.14 (m, 1H), 4.23-4.26 (m, 3H)□5.00 (s, 1H), 5.15(d, J = 1.6 Hz, 1H), 6.02-6.15 (m, 1H), 6.29 (d, J =4.0 Hz, 1H), 7.11-7.15 (m, 2H), 7.42 (d, J = 12.8Hz, 1H), 7.53 (t, J = 8.8 Hz, 2H) 11.35 (s, 1H); MS,m/e 601.92 (M + 1)⁺; |
| 75 | 4-Br-Ph | H | H | i-Bu (Leu) | Cyc-pent | δ 0.65-0.75 (m, 2H), 0.78-0.83 (m, 4H), 1.35-1.41(m, 3H), 1.51-1.60 (m, 6H), 1.71-1.77 (m, 5H), 3.51-3.67 (m, 1H), 4.11-4.15 (m, 2H), 4.23-4.28 (m,2H)□5.00 (m, 1H), 5.13 (d, J = 16.4 Hz, 1H), 6.03-6.09 (m, 1H), 6.29 (d, J = 5.6 Hz, 1H), 7.11 (t, J =16.8 Hz, 2H), 7.40-7.45 (m , 1H), 7.54 (t, J = 12.0Hz, 2H), 11.5 (s, 1H); MS, m/e 643.98 (M + 1)⁺; |
| 76 | 4-F-Ph | H | H | Et | Cyc-hex | δ 0.72-0.83 (m, 3H), 1.22-1.36 (m, 5H), 1.45-1.64(m, 5H), 1.65-1.73 (m, 5H), 3.58-3.60 (m, 1H), 4.12-4.27 (m, 4H), 4.62-4.64 (m, 1H) 5.12-5.16 (m, 1H),5.97-6.03 (m, 1H), 6.28-6.30 (m, 1H), 7.17-720 (m,4H), 7.41-7.45 (m, 1H), , 11.34 (d, J = 8.0 Hz, , 1H);MS, m/e 569.92 (M + 1)⁺; |
| 77 | 4-Cl-Ph | H | H | Et | Cyc-hex | δ 0.70-0.83 (m, 3H), 1.20-1.32 (m, 6H), 1.44-1.76(m, 9H), 3.57-3.60 (m, 1H), 4.12-4.30 (m, 4H), 4.62(s, 1H), 5.14 (d, J = 13.2 Hz, 1H), 6.02-6.10 (m, 1H),6.28-6.29 (m, 1H), 7.19 (d, J = 8.8 Hz, 2H), 7.39-7.43 (m, 3H), 11.35 (s, 1H); Ms: m/e 585.90 (M + 1)⁺,608.08 (M + 23)⁺ |
| 78 | 4-Br-Ph | H | H | Et | Cyc-hex | δ 0.72-0.85 (m, 3H), 1.22-1.31 (m, 6H), 1.44-1.72(m, 9H), 3.57-3.58 (m, 1H), 4.11-4.27 (m, 4H), 4.60-4.62 (m, 1H), 5.13 (d, J = 13.6 Hz, 1H), 6.02-6.08(m, 1H), 6.28-6.29 (m, 1H), 7.13 (d, J = 8.4 Hz, 2H),7.43 (d, J = 12.4 Hz, 1H), 7.51-7.55 (m, 2H), 11.35(s, 1H); Ms: m/e 629.87 (M + 1)⁺ |
| 79 | Ph | H | H | Et | Cyc-hex | δ 0.72-0.82 (m, 3H), 1.27-1.34 (m, 5H), 1.53-1.72(m, 10H), 3.51-3.67 (m, 1H), 4.13-4.27 (m, 4H),4.57-4.68 (m, 1H)□5.13 (d, J = 14.8 Hz, 1H), 5.91-6.06 (m, 1H), 6.29 (d, J = 4.4 Hz, 1H), 7.14-7.17 (m,3H), 7.30-7.36 (m, 2H), 7.43 (d, J = 16.8 Hz,1H), 11.33 (d, J = 10.4 Hz, , 1H); MS, m/e 551.95(M + 1)⁺; |
| 80 | 4-F-Ph | H | H | i-Bu (Leu) | Cyc-hex | δ: 0.68 (d, J = 6.0 Hz, 3H), 0.75-0.84 (m, 3H), 1.28-1.44 (m, 9H), 1.61-1.70 (m, 4H), 1.72 (s, 3H), 3.61-3.72 (m, 1H), 4.13-4.27 (m, 4H), 4.62 (s, 1H), 5.15(d, J = 14.8 Hz, 1H), 5.95-6.13 (m, 1H), 6.29 (d, J =6.0 Hz, 1H), 7.15-7.20 (m, 4H), 7.40-7.46 (m, 1H),11.34 (d, J = 10.4 Hz, 1H); MS, m/e 598.07(M + 1)⁺; |
| 81 | 4-Cl-Ph | H | H | i-Bu (Leu) | Cyc-hex | δ: 0.60-0.78 (m, 6H), 1.21-1.37 (m, 9H), 1.55-1.66(m, 7H), 3.55-3.59 (m, 1H)□4.08-4.21 (m,4H)□4.52-4.56 (m, 1H), 5.05 (d, J = 14.8 Hz1H)□5.96-6.06 (m, 1H), 6.21-6.23 (m, 1H)□7.08-7.13 (m, 2H)□ 7.31-7.38 (m, 3H), 11.26 (d, J = 9.2Hz 1H); MS, m/e 614.04 (M + 1)⁺; |
| 82 | 4-Br-Ph | H | H | i-Bu (Leu) | Cyc-hex | δ: 0.67-0.69 (m, 2H), 0.75-0.84 (m, 4H), 1.24-1.45(m, 9H), 1.62-1.72 (m, 7H), 3.51-3.62 (m, 1H), 4.11-4.27 (m, 4H), 4.60 (s, 1H)□5.13 (d, J = 16.0 Hz,1H), 6.07-6.10 (m, 1H), 6.29 (d, J = 5.6 Hz, 1H),7.11 (t, J = 16.0 Hz, 2H), 7.43 (d, J = 18.4 Hz, 1H),7.54 (t, J = 12.0 Hz, 2H), 11.35 (s, 1H); MS, m/e657.15 (M + 1)⁺; |
| 83 | Ph | H | H | i-Bu (Leu) | Cyc-hex | δ: 0.64-0.89 (m, 6H), 1.28-1.39 (m, 9H), 1.58-1.67(m, 7H), 3.61 (s, br, 1H), 4.09-4.20 (m, 4H), 4.58 (s,br, 1H), 5.07-5.11 (m, 1H), 5.92-6.07 (m, 1H), 6.24(s, 1H), 7.10-7.12 (m, 3H), 7.29-7.42 (m, 3H); MS,m/e 580 (M + 1)⁺; |
| 84 | 4-F-Ph | H | H | Me | Cyc-hexyl | δ: 1.17-1.24 (m, 3H), 1.28-1.47 (m, 6H), 1.64-1.69(m, 4H), 1.72 (s, 3H), 3.72-3.79 (m, 1H), 4.10-4.29(m, 4H), 4.61-4.63 (m, 1H), 5.12-5.17 (m, 1H), 6.05-6.14 (m, 1H), 6.27-6.30 (m, 1H), 7.18-7.23 (m, 4H),7.45 (s, 1H), 11.36 (s, 1H); MS, m/e 556.00 (M + 1)⁺; |
| 85 | 4-F-Ph | H | H | Me | Cyc-pentyl | δ: 1.15-1.21 (m, 3H), 1.51-1.59 (m, 6H), 1.71-1.78(m, 5H), 3.69-3.75 (m, 1H), 4.11-4.29 (m, 4H), 4.99-5.02 (m, 1H), 5.12-5.15 (d, J = 14.4 Hz, 1H), 6.03-6.09 (m, 1H), 6.28-6.30 (t, J = 3.8 Hz, 1H), 7.17-7.20(m, 4H), 7.41-7.45 (d, J = 16 Hz, 1H), 11.36 (s, 1H);MS, m/e 541.97 (M + 1)⁺/564.11 (M + 23)⁺; |
| 86 | 4-F-Ph | H | H | Me | Cyc-butyl | δ 1.19 (d, J = 18.8 Hz, 3H), 1.52-1.60 (m, 1H), 1.71(s, 3H), 1.62-1.75 (m, 1H), 1.88-1.99 (m, 2H), 2.18-2.27 (m, 2H), 3.70-3.82 (m, 1H), 4.10-4.30 (m, 4H),4.82-4.88 (m, 1H), 5.12-5.16 (m, 1H), 6.04-6.12 (m,1H), 6.27-6.31 (m, 1H), 7.18-7.23 (m, 4H), 7.45 (s,1H), 11.34 (s, 1H); MS, m/e 527.96 (M + 1)⁺; |
| 87 | 4-F-Ph | H | H | Me | Cyc-propylmethyl | δ: 0.20 (s, 2H), 0.49 (s, 2H), 1.02-1.06 (m, 1H),1.17-1.24 (m, 3H), 1.70-1.71 (t, J = 2.8 Hz, 3H), 3.81-3.85 (m, 3H), 4.11-4.29 (m, 4H), 5.11-5.15 (d, J = 16Hz, 1H), 6.10-6.14 (m, 1H), 6.26-6.29 (m, 1H), 7.17-7.22 (m, 4H), 7.40-7.44 (m, 1H), 11.36 (s, 1H); MS,m/e 527.96 (M + 1)⁺; |
| 88 | 4-Br-Ph | H | H | Me | Cyc-pentyl | δ: 1.18 (dd, J = 8.0 Hz, 8.4 Hz, 3H), 1.50-1.58 (m,6H), 1.70-1.72 (m, 5H), 3.62-3.75 (m, 1H), 4.10-4.14 (m, 1H), 4.23-4.26 (m, 3H)□5.00 (s, 1H), 5.15(d, J = 1.6 Hz, 1H), 6.02-6.15 (m, 1H), 6.29 (d, J =4.0 Hz, 1H), 7.11-7.15 (m, 2H), 7.42 (d, J = 12.8Hz, 1H), 7.53 (t, J = 8.8 Hz, 2H) 11.35 (s, 1H); MS,m/e 601.92 (M + 1)⁺; |
| 89 | 4-Br-Ph | H | H | Me | Cyc-butyl | δ: 1.13-1.18 (m, 3H), 1.49-1.57 (m, 1H), 1.66-1.68(m, 4H), 1.85-1.88 (m, 2H), 2.17-2.18 (m, 2H), 3.68-4.73 (m, 1H), 4.07-4.25 (m, 4H), 4.38-4.41 (m, 1H),5.10 (d, J = 14.8 Hz 1H), 6.07-6.11 (m, 1H), 6.24-6.26(m, 1H), 7.07-7.12 (m, 2H), 7.39 (dd, J = 11.6 1.6 Hz1H), 7.49-7.52 (m, 2H), 11.31 (s, 1H); MS, m/e589.90 (M + 1)⁺; |
| 90 | 4-Br-Ph | H | H | Me | Cyc-propylmethyl | δ: 0.23-0.25 (m, 2H), 0.47-0.50 (m, 2H), 0.98-1.10(m, 1H), 1.20-1.25 (m, 3H), 1.71-1.73 (m, 3H), 3.77-3.87 (m, 3H), 4.11-4.30 (m, 4H), 5.12-5.16 (m, 1H),6.14-6.17 (m, 1H), 6.27-6.30 (m, 1H), 7.12-7.16 (m,2H), 7.41-7.44 (m, 1H), 7.53-7.57 (m, 2H), 11.35 (s,1H); MS, m/e 587.93 (M + 1)⁺; |
| 91 | 4-Cl-Ph | H | H | Me | Cyc-hexyl | δ: 1.16-1.33 (m, 8H), 1.42-1.46 (m, 1H), 1.59-1.71(m, 7H), 3.71-3.79 (m, 1H), 4.11-4.27 (m, 4H), 4.54-4.59 (m, 1H), 5.12 (d, J = 14.0 Hz 1H), 6.07-6.13 (m,1H), 6.27-6.28 (m, 1H), 7.18 (t, J = 16.0 Hz 1H), 7.38-7.42 (m, 3H), 11.31 (s, 1H); MS, m/e 571.98(M + 1)⁺; |
| 92 | 4-Cl-Ph | H | H | Me | Cyc-pentyl | δ: 1.18 (dd, J = 12.4 Hz, 7.6 Hz, 3H), 1.51-1.60 (m,6H), 1.71-1.77 (m, 5H), 3.68-3.80 (m, 1H), 4.11-4.15 (m, 1H), 4.20-4.28 (m, 3H), 5.01 (t, J = 5.6 Hz,1H), 5.14 (d, J = 15.6 Hz, 1H), 6.09 (s, 1H), 6.28 (t,J = 6.0 Hz, 1H), 7.17-7.20 (m, 2H), 7.39-7.43 (m,3H), 11.35 (s, 1H); MS, m/e 557.96 (M + 1)⁺; |
| 93 | 4-Cl-Ph | H | H | Me | Cyc-butyl | δ: 1.15-1.21 (m, 3H), 1.50-1.60 (m, 1H), 1.69-1.70(m, 4H), 1.89-1.93 (m, 2H), 2.19-2.22 (m, 2H), 3.77-3.88 (m, 1H), 4.11-4.29 (m, 4H), 4.80-4.90 (m, 1H),5.12-5.14 (d, J = 14.8 Hz, 1H), 6.11 (s, 1H), 6.27-6.28(t, J = 3 Hz, 1H), 7.15-7.19 (m, 2H), 7.38-7.42 (m,3H), 11.36 (s, 1H); MS, m/e 543.97 (M + 1)⁺; |
| 94 | 4-Cl-Ph | H | H | Me | Cyc-propylmethyl | δ: 0.23-0.24 (m, 2H), 0.46-0.50 (m, 2H), 1.02-1.06 (m, 1H), 1.19-1.25 (m, 3H), 1.71-1.72 (m, 3H),3.83-3.87 (m, 3H), 4.11-4.16 (m, 1H), 4.23-4.27 (m,3H), 5.14 (d, J = 15.6 Hz 1H), 6.11-6.18 (m, 1H),6.27-6.30 (m, 1H), 7.17-7.22 (m, 2H), 7.40-7.44 (m,3H), 11.37 (s, 1H); MS, m/e 543.93 (M + 1)⁺; |
| 95 | Ph | H | H | Me | Cyc-butyl | δ: 1.15-1.21 (m, 3H), 1.50-1.60 (m, 1H), 1.63-1.70(m, 4H), 1.89-1.93 (m, 2H), 2.19-2.22 (m, 2H), 3.75-3.80 (m, 1H), 4.11-4.29 (m, 4H), 4.81 (s, 1H), 5.07-5.12 (t, J = 8.4 Hz, 1H), 6.11 (s, 1H), 6.23-6.26 (m,1H), 7.10-7.14 (m, 3H), 7.30-7.33 (m, 2H), 7.37-7.41 (d, d, J₁ = 14.4 Hz, J₂ = 1.2 Hz, 1H), 11.36 (s,1H); MS, m/e 509.9 (M + 1)⁺; |
| 96 | Ph | H | H | Me | Cyc-propylmethyl | δ: 0.23-0.25 (m, 2H), 0.46-0.50 (m, 2H), 1.02-1.06(m, 1H), 1.18-1.24 (m, 3H), 1.70-1.72 (m, 3H), 3.77-3.88 (m, 1H), 4.11-4.29 (m, 4H), 5.12-5.16 (m, 1H),6.05-6.16 (m, 1H), 6.27-6.30 (m, 1H), 7.14-7.21 (m,3H), 7.32-7.38 (m, 2H), 7.42-7.46 (m, 3H), 11.36 (s,1H); MS, m/e 509.97 (M + 1)⁺; |
| 97 | Ph | H | H | Me | —CH₂CF₃ | δ: 1.20-1.26 (d, d, J₁ = 20 Hz, J₂ = 7.2 Hz, 3H), 1.68-1.69 (d, J₁ = 6.4 Hz, 3H), 3.92-3.94 (d, J₁ = 10.4 Hz,1H), 4.11-4.29 (m, 4H), 4.70-4.74 (m, 2H), 5.10-5.14(d, J = 16 Hz, 1H), 6.24-6.28 (m, 2H), 7.13-7.16 (m,3H), 7.30-7.42 (m, 3H), 11.36 (s, 1H); MS, m/e538.1 (M + 1)⁺; |
| 98 | 4-F-Ph | H | H | Me | —CH₂CF₃ | δ: 1.22 (dd, J = 23.2 7.2 Hz 3H), 1.66 (d, J = 5.6 Hz3H), 3.89-3.94 (m, 1H), 4.05-4.24 (m, 4H), 4.65-4.78 (m, 2H), 5.09 (d, J = 14 Hz 1H), 6.19-6.25 (m,2H), 7.10-7.20 (m, 4H), 7.37 (d, J = 15.6 Hz 1H),11.37 (d, J = 6.8 Hz 1H); MS, m/e 555.93 (M + 1)⁺; |
| 99 | 4-Br-Ph | H | H | Me | —CH₂CF₃ | δ: 1.22-1.32 (m, 3H), 1.69-1.75 (m, 3H), 3.90-4.10(m, 1H), 4.12-4.29 (m, 4H), 4.72-4.79 (m, 2H), 5.11-5.15 (m, 1H)□6.28-6.35 (m, 2H), 7.15 (d, J = 6.8 Hz,2H), 7.39-7.42 (m, 1H), 7.52-7.58 (m, 2H), 11.35 (s,1H); MS, m/e 617.87 (M + 2)⁺; |
| 100 | Ph | H | H | Me | 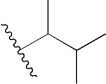 | δ: 0.83-0.99 (m, 6H), 1.05-1.09 (m, 3H), 1.14-1.32(m, 3H), 1.70-1.79 (m, 4H), 3.61-3.83 (m, 1H), 4.09-4.28 (m, 4H), 4.57-4.62 (m, 1H), 5.14 (d, J = 17.2Hz, 1H), 5.99-6.09 (m, 1H) 6.29 (s, 1H), 7.14-7.18(m, 3H), 7.32-7.44 (m, 3H), 11.34 (s, 1H); MS, m/e526.1 (M + 1)⁺; |
| 101 | Ph | H | H | Me | 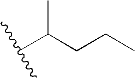 | δ: 0.79-0.90 (m, 3H), 1.20-1.28 (m, 3H), 1.38-1.45(m, 5H), 1.46-1.48 (m, 2H), 1.70-1.75 (m, 3H), 3.70-3.72 (m, 1H), 4.11-4.15 (m, 1H), 4.19-4.29 (m, 3H),4.76-4.79 (m, 1H), 5.14 (d, J = 14.8 Hz, 1H), 6.01-6.07 (m, 1H), 6.27-6.30 (m, 1H), 7.14-7.18 (m, 3H),7.32-7.37 (m, 2H), 7.41-7.45 (m, 1H), 11.33 (s, 1H);MS, m/e 526.16 (M + 1)⁺; |
| 102 | Ph | H | H | Me | 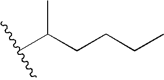 | δ: 0.80-0.83 (m, 3H), 1.10-1.23 (m, 9H), 1.37-1.47(m, 3H), 1.71 (s, 3H), 3.71-3.73 (m, 1H), 4.12-4.28(m, 4H), 4.75-4.78 (m, 1H), 5.12-5.16 (m, 1H), 6.03-6.06 (m, 1H), 6.28-6.30 (m, 1H), 7.15-7.18 (m, 3H),7.32-7.35 (m, 2H), 7.42-7.45 (m, 1H), 11.33 (s, 1H);MS, m/e 540.05 (M + 1)⁺; |
| 103 | Ph | H | H | Me | 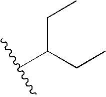 | δ: 0.76-0.81 (m, 6H), 1.20-1.26 (m, 3H), 1.43-1.52(m, 4H), 1.72 (s, 3H), 3.70-3.85 (m, 1H), 4.12-4.29(m, 4H), 4.62-4.64 (m, 1H), 5.12-5.16 (m, 1H), 6.04-6.07 (m, 1H), 6.29-6.30 (m, 1H), 7.14-7.17 (m, 3H),7.32-7.34 (m, 2H), 7.42-7.45 (m, 1H), 11.33 (s, 1H);MS, m/e 526.03 (M + 1)⁺; |
| 104 | Ph | H | H | Me | 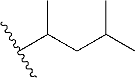 | δ: 0.75-0.81 (m, 6H), 1.03-1.19 (m, 7H), 1.41-1.47(m, 2H), 1.67-1.69 (m, 3H), 3.68-3.74 (m, 1H), 4.11-4.23 (m, 4H), 4.78-4.81 (m, 1H), 5.12 (d, J = 11.6 Hz1H), 5.95-6.04 (m, 1H), 6.26 (t, J = 3.2 Hz 1H), 7.11-7.15 (m, 3H), 7.30-7.34 (m, 2H), 7.43 (d, J = 13.6 Hz1H), 11.32 (d, J = 4.4 Hz 1H); MS, m/e 540.04(M + 1)⁺; |
| 105 | Ph | H | H | Me | 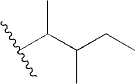 | δ: 0.76-0.80 (m, 6H), 1.00-1.07 (m, 4H), 1.18 (dd, J =16.8 Hz J = 7.2 Hz, 3H), 1.23-1.45 (m, 2H), 1.68(d, J = 4.4 Hz, 3H), 3.68-3.78 (m, 1H), 4.10-4.22 (m,4H), 4.63-4.75 (m, 1H), 5.10 (d, J = 14.8 Hz, 1H),5.95-6.08 (m, 1H), 6.24-6.27 (m, 1H), 7.11-7.15 (m,3H), 7.29-7.34 (m, 2H), 7.40 (d, J = 13.2 Hz, 3H),11.31 (d, J = 8.8 Hz, 1H); MS, m/e 543.93 (M + 1)⁺; |
| 106 | Ph | H | H | Me | 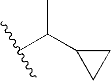 | δ: 0.24 (d, J = 3.2 Hz, 2H), 0.43-0.47 (m, 2H)□0.80-1.15 (m, 1H), 1.16-1.23 (m, 6H), 1.70-1.72 (m, 3H),3.68-3.85 (m, 1H), 4.14-4.27 (m, 5H), 5.16 (d, J =1.2 Hz, 1H), 6.00-6.15 (m, 1H), 6.27-6.30 (m, 1H),7.15-7.18 (m, 3H), 7.32-7.35 (m, 2H), 7.36-7.46 (m,1H), 11.36 (m, 1H); MS, m/e 546.19 (M + 23)⁺; |
| 107 | Ph | H | H | Me | 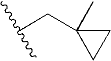 | δ: 0.29-0.33 (m, 2H), 0.43-0.45 (m, 2H), 1.41 (d, J =5.2 Hz, 3H), 1.24 (dd, J = 18 Hz J = 7.2 Hz, 3H),1.72 (d, J = 8.4 Hz, 3H), 3.77-3.90 (m, 3H), 4.09-4.29 (m, 1H), 5.14 (d, J = 17.2 Hz, 1H), 6.05-6.16(m, 1H), 6.28-6.30 (m, 1H), 7.15-7.29 (m, 3H), 7.33-7.38 (m, 2H), 7.43 (d, J = 16.4 Hz, 3H), 11.34 (d, J =8.4 Hz, 1H); MS, m/e 524.04 (M + 1)⁺; |
| 108 | Ph | H | H | Me | 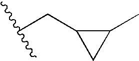 | δ: 0.20-0.30 (m, 1H), 0.35-0.45 (m, 1H), 0.66-0.67(m, 1H), 0.75-0.77 (m, 1H), 0.94-0.97 (m, 3H), 1.17-1.24 (m, 3H), 1.70-1.72 (m, 3H), 3.78-3.88 (m, 3H),4.11-4.29 (m, 4H), 5.13 (d, J = 15.6 Hz, 1H), 6.06-6.07 (m, 1H)□6.27-6.30 (m, 1H), 7.17 (t, J =13.6 Hz, 3H), 7.32-7.45 (m, 3H), 11.34 (d, J = 8.2 Hz,1H); MS, m/e 524.11 (M + 1)⁺; |
| 109 | Ph | H | H | Me | 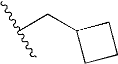 | δ: 1.21 (dd, J = 7.2 Hz, 18.4 Hz, 3H), 1.68-1.72 (m,5H), 1.80-1.81 (m, 2H), 1.94-1.95 (m, 2H), 2.51 (s,1H), 3.75-3.90 (m, 1H), 3.94-4.01 (m, 2H), 4.14-4.26 (m, 4H), 5.16 (d, J = 1.2 Hz, 1H), 6.00-6.15 (m,1H), 6.27-6.30 (m, 1H), 7.15-7.18 (m, 3H), 7.32-7.45 (m, 3H), 11.34 (d, J = 9.2 Hz, 1H); MS, m/e524.10 (M + 1)⁺; |
| 110 | Ph | H | H | Me | 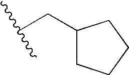 | δ: 1.12-1.28 (m, 5H), 1.46-1.55 (m, 4H), 1.62-1.71(m, 5H), 2.09 (s, 1H), 3.82-3.92 (m, 3H), 4.11-4.28(m, 4H), 5.13 (d, J = 15.6 Hz, 1H), 6.05-6.15 (m,1H), 6.27-6.30 (m, 1H), 7.16 (t, J = 12.8 Hz, 3H),7.32-7.45 (m, 3H), 11.34 (d, J = 9.2 Hz, 1H); MS,m/e 538.17 (M + 1)⁺; |
| 111 | Ph | H | H | Me | 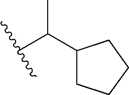 | δ: 1.12-1.23 (m, 8H), 1.45-1.49 (m, 4H), 1.50-1.52(m, 2H), 1.63 (s, 3H), 1.71-1.72 (m, 1H), 3.65-3.72(m, 1H), 4.14-4.26 (m, 4H), 4.58-4.70 (m, 1H), 5.12-5.16 (m, 1H), 6.01-6.10 (m, 1H), 6.28-6.29 (m, 1H),7.15-7.18 (m, 3H), 7.32-7.38 (m, 2H), 7.42-7.45 (m,1H), 11.45 (s, 1H); MS, m/e 552.12 (M + 1)⁺; |
| 112 | Ph | H | H | Me | 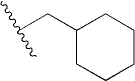 | δ: 0.80-0.92 (m, 2H), 1.10-1.23 (m, 6H), 1.58-1.72(m, 9H), 3.78-3.85 (m, 3H), 4.12-4.26 (m, 4H), 5.13(d, J = 15.6 Hz, 1H), 6.05-6.20 (m, 1H), 6.27-6.30(m, 1H), 7.14-7.18 (m, 3H), 7.32-7.45 (m, 3H),11.34 (d, J = 8.0 Hz, 1H); MS, m/e 552.14 (M + 1)⁺; |
| 113 | Ph | H | H | Me | 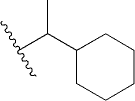 | δ: 0.80-0.99 (m, 2H), 1.08-1.12 (m, 5H), 1.15-1.23(m, 4H), 1.41 (s, 1H), 1.57-1.60 (m, 2H), 1.64-1.71(m, 6H), 3.70-3.85 (m, 1H), 4.11-4.28 (m, 4H), 4.58-4.60 (m, 1H), 5.13 (d, J = 14.8 Hz, 1H), 6.03-6.06(m, 1H)□6.27-6.30 (m, 1H), 7.16 (t, J = 14.4 Hz,3H), 7.32-7.44 (m, 3H), 11.34 (d, J = 8.2 Hz, 1H);MS, m/e 566.18 (M + 1)⁺; |
| 114 | Ph | H | H | Me | 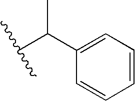 | δ: 1.14-1.27 (m, 4H), 1.41-1.44 (m, 3H), 1.70 (t, J =16.0 Hz, 3H), 3.70-3.92 (m, 1H), 4.11-4.26 (m, 4H),5.10-5.11 (m, 1H), 5.74-5.77 (m, 1H), 6.02-6.20 (m,1H), 6.28-6.29 (m, 1H), 7.07-7.18 (m, 3H), 7.28-7.45 (m, 8H), 11.35 (t, J = 10.8 Hz, 1H); MS, m/e559.96 (M + 1)⁺; |
| 115 | Ph | H | H | Me | 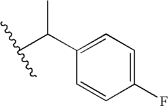 | δ: 1.16-1.26 (m, 3H), 1.41-1.44 (m, 3H), 1.70 (t, J =8.0 Hz, 3H), 3.72-3.79 (m, 1H), 4.11-4.27 (m, 4H),5.15-5.26 (m, 1H), 5.75-5.78 (m, 1H), 6.02-6.16 (m,1H), 6.27-6.29 (m, 1H), 7.06-7.18 (m, 5H), 7.29-7.46 (m, 5H), 11.34 (s, 1H); MS, m/e577.91 (M + 1)⁺; |
| 116 | Ph | H | H | i-Bu (Leu) | 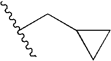 | δ: 0.24-0.28 (m, 2H), 0.46-0.48 (m, 2H), 0.68-0.0.70(m, 2H), 0.75-0.84 (m, 5H), 1.13-1.23 (m, 1H), 1.38-1.40 (m, 2H), 1.70-1.72 (m, 3H), 3.65-3.78 (m, 1H),3.82-3.86 (m, 2H), 4.14-4.30 (m, 4H), 5.16 (d, J =1.2 Hz, 1H), 5.95-6.13 (m, 1H), 6.27-6.30 (m, 1H),7.12-7.18 (m, 3H), 7.32-7.46 (m, 3H), 11.36 (m, 1H);MS, m/e 552.15 (M + 1)⁺; |
| 117 | Ph | H | Me | H | 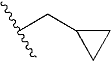 | δ: 0.22-0.23 (m, 2H), 0.47 (d, J = 7.2 Hz, 2H), 1.04(s, 1H), 1.19-1.22 (m, 3H), 1.72 (s, 3H), 3.79-3.90(m, 3H), 4.10-4.26 (m, 4H), 5.14 (d, J = 15.2 Hz,1H), 6.03-6.12 (m, 1H), 6.28 (s, 1H), 7.14-7.20 (m,3H)□7.32-7.44 (m, 3H), 11.33 (d, J = 5.2 Hz, 1H);MS, m/e 510.06 (M + 1)⁺; |
| 118 | Ph | H | Me | H | 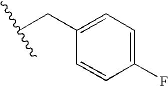 | δ: 1.18-1.22 (m, 3H), 1.68 (d, J = 7.6 Hz, 3H), 3.83-3.87 (m, 1H), 4.09-4.26 (m, 4H), 4.99-5.11 (m, 3H),6.07-6.17 (m, 1H), 6.26-6.27 (m, 1H), 7.09-7.18 (m,5H), 7.29-7.42 (m, 5H), 11.33 (s, 1H); MS, m/e 564.1(M + 1)⁺; |
| 119 | Ph | H | Me | H | 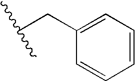 | δ: 1.19-1.25 (m, 3H), 1.70 (d, J = 11.2 Hz, 3H), 3.85-3.89 (m, 1H), 4.10-4.27 (m, 4H), 5.02-5.13 (m, 3H),6.09-6.19 (m, 1H), 6.28 (d, J = 5.6 Hz, 1H), 7.11-7.18 (m, 3H), 7.31-7.43 (m, 8H), 11.33 (s, 1H); MS,m/e 546.1 (M + 1)⁺; |
| 120 | 4-FPh | H | Me | Me | Me | δ: 1.23-1.46 (m, 6H), 1.72 (d, J = 14.4 Hz□3H),3.46-3.60 (m, 3H), 4.11-4.28 (m, 4H), 5.15 (s, 1H),5.97 (d, J = 10.0 Hz, 1H), 6.30 (d, J = 4.8 Hz, 1H),7.10-7.43 (m, 4H), 7.71 (s, 1H), 11.34 (d, J = 5.6 Hz,1H); MS, m/e 501.99 (M + 1)⁺; |
| 121 | Ph | H | # | # | Me | δ: 0.89-0.96 (m, 1H), 1.04-1.10 (m, 1H), 1.22-1.26(m, 2H), 1.70 (s, 3H), 3.54 (s, 3H), 4.11-4.15 (m,1H), 4.26-4.28 (m, 3H), 5.15 (s, 1H), 6.29 (t, J =6.0 Hz 1H)□6.54 (d, J = 16.0 Hz, 1H), 7.13-7.19 (m,3H), 7.33-7.38 (m, 2H), 7.45 (s, 1H), 11.33 (d, J =7.6 Hz, 1H); MS, m/e 482.0 (M + 1)⁺; |

*R² and R^{3b} connect N and C_{α}-carbon via —(CH₂)₃—. ;
# R^{3a} and R^{3b} linked with —(CH₂)₂—.

Biological Screening Methods

HIV Activity:

1-HIV Screen: Primary Screening of PSI Compounds are Tested for Antiviral HIV Activity at 50 μM. The cells used are P4CCR5luc cells; they are human HIV indicator cells, which are derived from Hela cells, express CD4, CXCR4, CCR5, luciferase, and a beta-gal gene under the control of HIV-1 LTR. P4CCR5 luc cells are cultivated in DMEM, 10% FBS, Penicillin, Streptomycin, and G418 at 500 μg/ml. 100 ul of P4 CCR5-luc cells are plated at 10,000 cells per well in 96 well Opaque Assay plates and incubated overnight at 37° C. The next day, the media is aspirated from the plates and replaced by 100 μL of compound freshly diluted into media at 2×50 μM, in triplicate, for 4 hours at 37° C. The cells are then infected with 100 μL NL43 virus at 5 ng of p24 per well, in the presence of 2×20 μg/mL of DEAE-Dextran for 40-42 hours. Non infected, infected no drug and AZT controls are always present in triplicate on each plate. After infection the beta-gal is quantitated using the Galacto-Star kit from Applied Biosystems using the manufacturer instructions and the luminescence measured using a Victor apparatus from Perkin-Elmer. Results are represented as percentage inhibition compare to untreated cells. The assays are performed in 2 to 3 independent experiments.

2-Titration of PSI Activity to Determine EC₅₀ on P4 CCR-luc Cells.

P4 CCR5-luc cells are plated at 10,000 cells per well (100 μL) in 96 well Opaque Assay plates and incubated overnight at 37° C. The next day, the media is aspirated from the plates and replaced by 100 ul of compound freshly diluted into appropriate media (DMEM, 10% FBS, G418 500 μg/mL, penicillin/streptomycin) at 2× final concentrations in 5 fold dilutions, usually from 2×100 μM to 2×0.032 μM, in triplicate, for 4 hours at 37° C. The cells are then infected with 100 μL NL43 wild type or mutant virus, at 5 ng to 20 ng of p24 per well, in the presence of 2×20 μg/mL of DEAE-Dextran, for 40-42hours. Non infected and infected no drug controls are always present in 12 plicate on each plate. An AZT control is tested in parallel for each experiment. After infection, the beta-gal is quantitated in the cell lysate using the Galacto-Star kit from Applied Biosystems and the luminescence measured using a Victor apparatus from Perkin-Elmer. The EC₅₀ (Effective Concentration) is calculated using a Microsoft® Excel® spreadsheet that calculates the concentration necessary to inhibit the 50% of the infection. The assay is performed in at least 2 independent experiments.

Toxicity

1-Luciferase Assay

P4 CCR5-luc cells are plated at 10,000 cells per well (100 μL) in 96 well Opaque Assay plates and incubated overnight at 37° C. The next day, the media is aspirated from the plates and replaced by 200 μL of compound freshly diluted into media in 5 fold dilutions from 100 μM to 0.0062 μM. After 4 days of incubation at 37° C., the luciferase activity is measured in the cell lysate using the Bright Glow kit from Promega and the luminescence measured using a Victor apparatus from Perkin-Elmer.

2-MTS Assays

Human cells lines Huh 7 and HepG2 (liver), BxPC3 (pancreatic) and CEM (lymphoid) are used for the MTS assays in 96 wells plates. Drugs are freshly diluted in media at 2×100 μM, 50 μM, 25 μM, 10 μM, 5 μM, 1 μM and 50 μL is dispensed in triplicate in the plates. The wells at the periphery of the plate contain 100 ul of media only and will be the blank controls. A 6 plicate control with no drug is always performed in each plate. 50 ul of cells are added to the plate, at 2000 cells per well for Huh 7, HepG2 and PxPC3, and 5000 cells per well for CEM cells. No cells are added at the periphery of the plate. The media used for Huh-7, HepG2 and BxPc3 cells is DMEM with 10% FBS, and Penicillin/streptomycin, and RPMI with 10% FBS, and Penicillin/streptomycin for CEM cells. After 8 days of incubation at 37 C, 20 μL of MTS dye from the CellTiter 96 Aqueous One Solution Cell Proliferation Assay kit from Promega is added to each well and the plate incubated for 2 h at 37° C. The absorbance is then read at 490 nm using the microplate reader EL1800 from Biotek. The signal is calculated by subtracting the absorbance measured in the blank controls. The CC₅₀ (Cytotoxic Concentration) value is then determined by comparing the signal obtained with the no-drug cell control with the treated cells and calculating the concentration of drug necessary to inhibit 50% of the signal in the wells treated with drugs.

Biological screening results are listed in Table 2 below.

TABLE 2
Screening Results

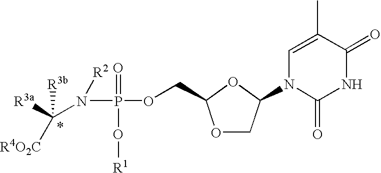

| | | | | | | | Toxicity |
|---|---|---|---|---|---|---|---|
| | | | | | | EC₅₀ (P4) | (Luciferase, |
| Exs. | R¹ | R² | R^{3a} | R^{3b} | R⁴ | (WT, μM) | 6d) |
| DOT | | | | | | 6.5 | >100 |
| 17 | Ph | H | H | Me | pentyl | 0.36 | >100 |
| 18 | Ph | H | H | Me | hexyl | 0.74 | >100 |
| 19 | Ph | H | H | Me | 4-F-Bn | 0.195 | >100 |
| 26 | 4-Br-Ph | H | H | Me | Et | 0.23 | >100 |
| 52 | Ph | H | H | i-Bu (Leu) | Me | 0.96 | >100 |
| 67 | 4-F-Ph | H | Me | H | Bn | 1.11 | >100 |
| 72 | Ph | H | H | Me | Cyc-hex | 2.12 | >100 |
| 73 | Ph | H | H | Me | Cyc-pent | 1.03 | >100 |
| 86 | 4-F-Ph | H | H | Me | Cyc-butyl | 0.50 | >100 |
| 95 | Ph | H | H | Me | Cyc-butyl | 0.45 | >100 |
| 96 | Ph | H | H | Me | Cyc-propylmethyl | 0.33 | >100 |
| 109 | Ph | H | H | Me | Cyclobutylmethyl- | 0.71 | >100 |
| 116 | Ph | H | H | i-Bu (Leu) | Cyclopropyl- | 0.57 | >100 |
| | | | | | methyl | | |
| 119 | Ph | H | Me | H | CH₂Ph | 1.70 | >100 |

Comparison Compounds^{a}

| Comparison | | | | | | EC₅₀ (P4) | Toxicity |
|---|---|---|---|---|---|---|---|
| Compd #^{b} | R¹ | R² | R^{3a} | R^{3b} | R⁴ | (WT, μM) | (Luciferase, 6d) |
| 28 | 1-Napth | H | H | Me | CH₂Ph | 1.34 | >100 |
| 20 | 4-Br-Ph | H | H | Me | Me | 0.27 | >100 |
| 25 | 2,4-diCl-Ph | H | H | Me | Me | 0.26 | >100 |
| 19 | 4-F-Ph | H | H | Me | Me | 0.99 | >100 |
| 18 | 4-Cl-Ph | H | H | Me | Me | 0.41 | ~100 |
| 26 | 1-Napth | H | H | Me | Me | 0.57 | 40.5 |
| 5 | Ph | H | H | Me | Me | 0.87 | >100 |
| 9 | Ph | H | H | iPr | Me | >100 | >100 |

^{a}Comparison compounds disclosed in Liang et al. Bioorg. Med. Chem. 2006, 14, 2178-2189.
^{b}Compound numbers correspond to numbered compound disclosed in Liang et al.

The present application claims priority to U.S. provisional patent application 60/979,961, filed Oct. 15, 2007, the contents of which are incorporated by reference in its entirety.

We claim:

1. A compound, or its pharmaceutically acceptable salt, of the formula: 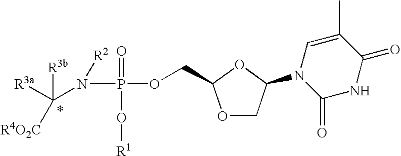
wherein:
R¹ is hydrogen, n-alkyl, branched alkyl, substituted or unsubstituted cycloalkyl, or aryl, which includes, but is not limited to, phenyl or naphthyl,
where phenyl or naphthyl is optionally substituted with at least one of C₁₋₆ alkyl, C₂₋₆ alkenyl, C₂₋₆ alkynyl, C₁₋₆ alkoxy, F, Cl, Br, I, nitro, cyano, C₁₋₆ haloalkyl, —N(R^{1′})₂, C₁₋₆ acylamino, —NHSO₂C₁₋₆ alkyl, —SO₂N(R^{1′})₂, COR^{1″}, and —SO₂C₁₋₆ alkyl,
where R^{1′} is independently hydrogen or alkyl, which includes, but is not limited to, C₁₋₂₀ alkyl, C₁₋₁₀ alkyl, or C₁₋₆ alkyl, and R^{1″} is —OR′ or —N(R^{1′})₂;
R² is hydrogen, C₁₋₁₀ alkyl, either R^{3a} and R² or R^{3b} and R² together are (CH₂)ₙ so as to form a cyclic ring that includes the adjoining N and C atoms, C(O)CR^{3a}R^{3b}NHR¹, where n is 2 to 4 and R¹, R^{3a}, and R^{3b} are as defined herein;
R^{3a} and R^{3b} are
(i) independently selected from hydrogen, C₁₋₁₂ alkyl (particularly when the alkyl is an amino acid residue), —(CH₂)_{c}(NR^{3′})₂, C₁₋₆ hydroxyalkyl, —CH₂SH, —(CH₂)₂S(O)_{d}Me, —(CH₂)₃NHC(═NH)NH₂, (1H-indol-3-yl)methyl, (1H-imidazol-4-yl)methyl, —(CH₂)ₑCOR^{3″}, aryl or aryl C₁₋₃ alkyl, said aryl groups optionally substituted with a group selected from hydroxyl, C₁₋₁₀ alkyl, C₁₋₆ alkoxy, halogen, nitro or cyano,
where c is 1 to 6, d is 0 to 2, and e is 0 to 3 and
R^{3′} is independently hydrogen or C₁₋₆ alkyl and R^{3″} is —OR^{3′} or —N(R^{3′})₂,
(ii) R^{3a} and R^{3b} both are C₁₋₆ alkyl,
(iii) R^{3a} and R^{3b} together are (CH₂)_{f} so as to form a spiro ring,
where f is 3 to 5,
(iv) R^{3a} is hydrogen and R^{3b} and R² together are (CH₂)ₙ so as to form a cyclic ring that includes the adjoining N and C atoms,
where n is 2 to 4,
(v) R^{3b} is hydrogen and R^{3a} and R² together are (CH₂)ₙ so as to form a cyclic ring that includes the adjoining N and C atoms,
where n is 2 to 4,
(vi) R^{3a} is H and R^{3b} is independently selected from H, CH₃, CH(CH₃)₂, CH₂CH(CH₃)₂, CH(CH₃)CH₂CH₃, CH₂Ph, (1H-indol-3-yl)methyl, (1H-imidazol-4-yl)methyl, —CH₂CH₂SCH₃, CH₂CO₂H, CH₂C(O)NH₂, CH₂CH₂COOH, CH₂CH₂C(O)NH₂, CH₂CH₂CH₂CH₂NH₂, —CH₂CH₂CH2NHC(NH)NH₂, CH₂OH, CH(OH)CH₃, CH₂((4′-OH)-Ph), or CH₂SH, or
(vii) R^{3a} is CH₃, CH(CH₃)₂, CH₂CH(CH₃)₂, CH(CH₃)CH₂CH₃, CH₂Ph, CH₂-indol-3-yl, —CH₂CH₂SCH₃, CH₂CO₂H, CH₂C(O)NH₂, CH₂CH₂COOH, CH₂CH₂C(O)NH₂, CH₂CH₂CH₂CH₂NH₂, —CH₂CH₂CH2NHC(NH)NH₂, CH₂-imidazol-4-yl, CH₂OH, CH(OH)CH₃, CH₂((4′-OH)-Ph), or CH₂SH and R^{3b} is H; and
R⁴ is hydrogen, C₁₋₁₀ alkyl, C₁₋₁₀ alkyl optionally substituted with a lower alkyl, alkoxy, substituted or unsubstituted cycloalkyl, halogen, C₁₋₁₀ haloalkyl, or substituted or unsubstituted aryl;
with the proviso that that the active compound represented by formula I is not selected from the group consisting of:

| | | | | | |
|---|---|---|---|---|---|
| (1) | R1 = 1-Napth | R2 = H | R3a = H | R3b = Me | R4 = CH₂Ph; |
| (2) | R1 = 4-Br-Ph | R2 = H | R3a = H | R3b = Me | R4 = Me; |
| (3) | R1 = 2,4-diCl-Ph | R2 = H | R3a = H | R3b = Me | R4 = Me; |
| (4) | R1 = 4-F-Ph | R2 = H | R3a = H | R3b = Me | R4 = Me; |
| (5) | R1 = 4-Cl-Ph | R2 = H | R3a = H | R3b = Me | R4 = Me; |
| (6) | R1 = 1-Napth | R2 = H | R3a = H | R3b = Me | R4 = Me; |
| (7) | R1 = Ph | R2 = H | R3a = H | R3b = Me | R4 = Me; |
| (8) | R1 = Ph | R2 = H | R3a = H | R3b = iPr | R4 = Me. |
| (9) | R1 = Ph | R2 = H | R3a = H | R3b = H | R4 = CH₃; |
| (10) | R1 = Ph | R2 = H | R3a = Me | R3b = Me | R4 = Me; |
| (11) | R1 = Ph | R2 = H | R3a = Me | R3b = H | R4 = Me; |
| (12) | R1 = Ph | R2 = H | R3a = H | R3b = CH₂Ph | R4 = Me; |
| (13) | R1 = Ph | R2 = H | R3a = CH₂Ph | R3b = H | R4 = Me; |
| (14) | R1 = Ph | R2 = H | R3a = iPr | R3b = H | R4 = Me; |
| (15) | R1 = Ph | R2 = H | R3a = H | R3b = Me | R4 = t-Bu; |
| (16) | R1 = Ph | R2 = H | R3a = H | R3b = Me | R4 = CH₂Ph; |
| (17) | R1 = 4-Me-Ph | R2 = H | R3a = H | R3b = Me | R4 = CH₃; |
| (18) | R1 = 4-Propyl-Ph | R2 = H | R3a = H | R3b = Me | R4 = Me; |
| (19) | R1 = 4-Neopent-Ph | R2 = H | R3a = H | R3b = Me | R4 = Me; |
| (20) | R1 = 4-MeO-Ph | R2 = H | R3a = H | R3b = Me | R4 = Me; |
| (21) | R1 = 4-CN-Ph | R2 = H | R3a = H | R3b = Me | R4 = Me; |
| (22) | R1 = 4-Br-Ph | R2 = H | R3a = H | R3b = Me | R4 = CH₂Ph; |
| (23) | R1 = 2-Cl-Ph | R2 = H | R3a = H | R3b = Me | R4 = Me; |
| (24) | R1 = 4-Cl-Ph | R2 = H | R3a = H | R3b = Me | R4 = CH₂Ph; |
| (25) | R1 = 2-Allyl-Ph | R2 = H | R3a = H | R3b = Me | R4 = Me; |
| (26) | R1 = 1-Napth | R2 = H | R3a = Me | R3b = Me | R4 = Me; |
| (27) | R1 = C₁₆H₃₃O(CH₂)₃ | R2 = H | R3a = H | R3b = H | R4 = Me; |
| (28) | R1 = C₁₆H₃₃O(CH₂)₃ | R2 = H | R3a = H | R3b = Me | R4 = Me; |
| (29) | R1 = C₁₆H₃₃O(CH₂)₃ | R2 = H | R3a = H | R3b = iPr | R4 = Me; |
| (30) | R1 = C₁₈H₃₇O(CH₂)₂ | R2 = H | R3a = H | R3b = Me | R4 = Me; and |
| (31) | R1 = Oleyl | R2 = H | R3a = H | R3b = Me | R4 = Me. |

2. A compound of the formula of claim 1 or its pharmaceutically acceptable salt that is selected from among.

| R¹ | R² | R^{3a} | R^{3b} | R⁴ |
|---|---|---|---|---|
| 4-Br-Ph | H | H | i-Pr (Val) | Me |
| Ph | H | H | Me | Et |
| Ph | H | H | Me | n-Bu |
| Ph | H | H | Me | 2-Bu |
| Ph | H | H | Me | iPr |
| Ph | H | H | CH₂Ph (Phe) | Et |
| 4-MeO-Ph | H | H | Me | Bn |
| 1-Napth | H | H | Me | Et |
| 3,4-di-Cl-Ph | H | H | Me | Me |
| Ph | H | H | H (Gly) | Et |
| Ph | H | Me | Me | Bn |
| Ph | H | H | H (Gly) | Bn |
| Ph | * | H | * | Me |
| | | | (Pro) | |
| Ph | H | H | Me | pentyl |
| Ph | H | H | Me | hexyl |
| Ph | H | H | Me | 4-F-Bn |
| 4-Cl-Ph | H | H | Me | Et |
| 4-Cl-Ph | H | H | Me | i-Pr |
| 4-Cl-Ph | H | H | Me | n-Bu |
| 4-Cl-Ph | H | H | Me | Bn |
| 2-Cl-Ph | H | H | Me | i-Pr |
| 2-Cl-Ph | H | H | Me | n-Bu |
| 2-Cl-Ph | H | H | Me | Bn |
| 4-Br-Ph | H | H | Me | Et |
| 4-Br-Ph | H | H | Me | i-Pr |
| 4-Br-Ph | H | H | Me | n-Bu |
| 4-Br-Ph | H | H | Me | hexyl |
| 4-Br-Ph | H | H | Me | propyl |
| 4-Br-Ph | H | H | Me | pentyl |
| 4-Br-Ph | H | H | Me | 2-Bu |
| 4-Br-Ph | H | H | Me | cyclo-hex |
| 4-Br-Ph | H | H | Me | t-Bu |
| 4-F-Ph | H | H | Me | Et |
| 4-F-Ph | H | H | Me | i-Pr |
| 4-F-Ph | H | H | Me | n-Bu |
| 4-F-Ph | H | H | Me | Bn |
| 2,4-di-Cl-Ph | H | H | Me | Et |
| 2,4-di-Cl-Ph | H | H | Me | i-Pr |
| 2,4-di-Cl-Ph | H | H | Me | n-Bu |
| 2,4-di-Cl-Ph | H | H | Me | Bn |
| 3,4-di-Cl-Ph | H | H | Me | Et |
| 3,4-di-Cl-Ph | H | H | Me | i-Pr |
| 3,4-di-Cl-Ph | H | H | Me | n-Bu |
| 3,4-di-Cl-Ph | H | H | Me | Bn |
| 4-MeO-Ph | H | H | Me | i-Pr |
| 4-MeO-Ph | H | H | Me | n-Bu |
| 4-Me-Ph | H | H | Me | i-Pr |
| 4-Me-Ph | H | H | Me | n-Bu |
| 4-Me-Ph | H | H | Me | Bn |
| Ph | H | H | i-Bu | Me |
| | | | (Leu) | |
| Ph | H | H | 3-indolyl-CH₂- | Me |
| | | | (Trp) | |
| Ph | H | H | Sec-Butyl (Ile) | Me |
| Ph | H | H | Methylmercapto- | Me |
| | | | Et (Met) | |
| 4-Br-Ph | H | H | i-Butyl(Leu) | Me |
| 4-Br-Ph | H | H | i-Bu (Leu) | Et |
| 4-Br-Ph | H | H | i-Bu (Leu) | i-Pr |
| 4-Br-Ph | H | H | i-Bu (Leu) | n-Bu |
| 4-Br-Ph | H | H | i-Bu (Leu) | Bn |
| 4-Br-Ph | H | Me | H | Me |
| 4-Br-Ph | H | Me | H | n-Bu |
| 4-Br-Ph | H | Me | H | Bn |
| 4-F-Ph | H | H | i-Bu (Leu) | Me |
| 4-F-Ph | H | H | i-Bu (Leu) | Bn |
| 4-F-Ph | H | Me | H | Me |
| 4-F-Ph | H | Me | H | Bn |
| 4-Cl-Ph | H | H | i-Bu (Leu) | Me |
| 4-Cl-Ph | H | H | i-Bu (Leu) | Bn |
| 4-Cl-Ph | H | Me | H | Me |
| 4-Cl-Ph | H | Me | H | Bn |
| Ph | H | H | Me | Cyc-hex |
| Ph | H | H | Me | Cyc-pent |
| 4-Br-Ph | H | H | Me | Cyc-pent |
| 4-Br-Ph | H | H | i-Bu (Leu) | Cyc-pent |
| 4-F-Ph | H | H | Et | Cyc-hex |
| 4-Cl-Ph | H | H | Et | Cyc-hex |
| 4-Br-Ph | H | H | Et | Cyc-hex |
| Ph | H | H | Et | Cyc-hex |
| 4-F-Ph | H | H | i-Bu (Leu) | Cyc-hex |
| 4-Cl-Ph | H | H | i-Bu (Leu) | Cyc-hex |
| 4-Br-Ph | H | H | i-Bu (Leu) | Cyc-hex |
| Ph | H | H | i-Bu (Leu) | Cyc-hex |
| 4-MeO-Ph | H | H | Me | Cyc-hex |
| 4-F-Ph | H | H | Me | Cyc-hexyl |
| 4-F-Ph | H | H | Me | Cyc-pentyl |
| 4-F-Ph | H | H | Me | Cyc-butyl |
| 4-F-Ph | H | H | Me | Cyc-propylmethyl |
| 4-Br-Ph | H | H | Me | Cyc-pentyl |
| 4-Br-Ph | H | H | Me | Cyc-butyl |
| 4-Br-Ph | H | H | Me | Cyc-propylmethyl |
| 4-Cl-Ph | H | H | Me | Cyc-hexyl |
| 4-Cl-Ph | H | H | Me | Cyc-pentyl |
| 4-Cl-Ph | H | H | Me | Cyc-butyl |
| 4-Cl-Ph | H | H | Me | Cyc-propylmethyl |
| Ph | H | H | Me | Cyc-butyl |
| Ph | H | H | Me | Cyc-propylmethyl |
| Ph | H | H | Me | —CH2CF3 |
| 4-F-Ph | H | H | Me | —CH2CF3 |
| 4-Br-Ph | H | H | Me | —CH2CF3 |
| Ph | H | H | Me | (1,2-Dimethyl-propyl) |
| Ph | H | H | Me | (1-Methyl-butyl) |
| Ph | H | H | Me | (1-Methyl-pentyl) |
| Ph | H | H | Me | (1-Ethyl-propyl) |
| Ph | H | H | Me | (1,3-Dimethyl-butyl)- |
| Ph | H | H | Me | (1,2-Dimethyl-butyl) |
| Ph | H | H | Me | (1-Cyclopropyl-ethyl) |
| Ph | H | H | Me | (1-Methyl- |
| | | | | cyclopropylmethyl) |
| Ph | H | H | Me | (2-Methyl- |
| | | | | cyclopropylmethyl) |
| Ph | H | H | Me | Cyclobutylmethyl- |
| Ph | H | H | Me | Cyclopentylmethyl- |
| Ph | H | H | Me | 1-Cyclopentyl-ethyl |
| Ph | H | H | Me | Cyclohexyylmethyl- |
| Ph | H | H | Me | 1-Cyclohexyl-ethyl |
| Ph | H | H | Me | 1-Phenyl-ethyl |
| Ph | H | H | Me | 1-(4-Fluoro-phenyl)-ethyl |
| Ph | H | H | i-Bu (Leu) | Cyclopropyl-methyl |
| Ph | H | Me | H | cyclopropyl-methyl |
| Ph | H | Me | H | 4-F-Ph-CH₂ |
| Ph | H | Me | H | CH₂Ph |
| 4-FPh | H | Me | Me | Me |
| Ph | H | # | # | Me |

Wherein for
* R₂ and R_{3b} connect N and Cα -carbon via —(CH₂)₃—;
# R₃ₐ and R_{3b} linked with —(CH₂)₂—.

3. A pharmaceutical composition that comprises an effective HIV treatment amount of a compound of claim 1 in a pharmaceutically acceptable carrier or diluent.

4. A method for the treatment of a host infected with HIV that includes administering an effective amount of a compound, or pharmaceutically acceptable salt, of the formula: 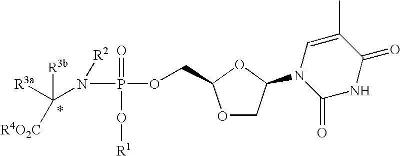
wherein:
R¹ is hydrogen, n-alkyl, branched alkyl, substituted or unsubstituted cycloalkyl, or aryl, which includes, but is not limited to, phenyl or naphthyl,
where phenyl or naphthyl is optionally substituted with at least one of C₁₋₆ alkyl, C₂₋₆ alkenyl, C₂₋₆ alkynyl, C₁₋₆ alkoxy, F, Cl, Br, I, nitro, cyano, C₁₋₆ haloalkyl, —N(R^{1′})₂, C₁₋₆ acylamino, —NHSO₂C₁₋₆ alkyl, —SO₂N(R^{1′})₂, COR^{1″}, and —SO₂C₁₋₆ alkyl,
where R^{1′} is independently hydrogen or alkyl, which includes, but is not limited to, C₁₋₂₀ alkyl, C₁₋₁₀ alkyl, or C₁₋₆ alkyl, and R^{1″} is —OR′ or —N(R^{1′})₂;
R² is hydrogen, C₁₋₁₀ alkyl, either R^{3a} and R² or R^{3b} and R² together are (CH₂)ₙ so as to form a cyclic ring that includes the adjoining N and C atoms, C(O)CR^{3a}R^{3b}NHR¹, where n is 2 to 4 and R¹, R^{3a}, and R^{3b} are as defined herein;
R^{3a} and R^{3b} are
(i) independently selected from hydrogen, C₁₋₁₂ alkyl (particularly when the alkyl is an amino acid residue), —(CH₂)_{c}(NR^{3′})₂, C₁₋₆ hydroxyalkyl, —CH₂SH, —(CH₂)₂S(O)_{d}Me, —(CH₂)₃NHC(═NH)NH₂, (1H-indol-3-yl)methyl, (1H-imidazol-4-yl)methyl, —(CH₂)ₑCOR^{3″}, aryl or aryl C₁₋₃ alkyl, said aryl groups optionally substituted with a group selected from hydroxyl, C₁₋₁₀ alkyl, C₁₋₆ alkoxy, halogen, nitro or cyano,
where c is 1 to 6, d is 0 to 2, and e is 0 to 3 and
R^{3′} is independently hydrogen or C₁₋₆ alkyl and R^{3″} is —OR^{3′} or —N(R^{3′})₂,
(ii) R^{3a} and R^{3b} both are C₁₋₆ alkyl,
(iii) R^{3a} and R^{3b} together are (CH₂)_{f} so as to form a spiro ring,
where f is 3 to 5,
(iv) R^{3a} is hydrogen and R^{3b} and R² together are (CH₂)ₙ so as to form a cyclic ring that includes the adjoining N and C atoms,
where n is 2 to 4,
(v) R^{3b} is hydrogen and R^{3a} and R² together are (CH₂)ₙ so as to form a cyclic ring that includes the adjoining N and C atoms,
where n is 2 to 4,
(vi) R^{3a} is H and R^{3b} is independently selected from H, CH₃, CH(CH₃)₂, CH₂CH(CH₃)₂, CH(CH₃)CH₂CH₃, CH₂Ph, (1H-indol-3-yl)methyl, (1H-imidazol-4-yl)methyl, —CH₂CH₂SCH₃, CH₂CO₂H, CH₂C(O)NH₂, CH₂CH₂COOH, CH₂CH₂C(O)NH₂, CH₂CH₂CH₂CH₂NH₂, —CH₂CH₂CH₂NHC(NH)NH₂, CH₂OH, CH(OH)CH₃, CH₂((4′-OH)-Ph), or CH₂SH, or
(vii) R^{3a} is CH₃, CH(CH₃)₂, CH₂CH(CH₃)₂, CH(CH₃)CH₂CH₃, CH₂Ph, CH₂-indol-3-yl, —CH₂CH₂SCH₃, CH₂CO₂H, CH₂C(O)NH₂, CH₂CH₂COOH, CH₂CH₂C(O)NH₂, CH₂CH₂CH₂CH₂NH₂, —CH₂CH₂CH₂NHC(NH)NH₂, CH₂-imidazol-4-yl, CH₂OH, CH(OH)CH₃, CH₂((4′-OH)-Ph), or CH₂SH and R^{3b} is H; and
R⁴ is hydrogen, C₁ alkyl, C₁₋₁₀ alkyl optionally substituted with a lower alkyl, alkoxy, substituted or unsubstituted cycloalkyl, halogen, C₁₋₁₀ haloalkyl, or substituted or unsubstituted aryl;
with the proviso that that the active compound represented by formula I is not selected from the group consisting of:

| | | | | | |
|---|---|---|---|---|---|
| (1) | R1 = 1-Napth | R2 = H | R3a = H | R3b = Me | R4 = CH₂Ph; |
| (2) | R1 = 4-Br-Ph | R2 = H | R3a = H | R3b = Me | R4 = Me; |
| (3) | R1 = 2,4-diCl-Ph | R2 = H | R3a = H | R3b = Me | R4 = Me; |
| (4) | R1 = 4-F-Ph | R2 = H | R3a = H | R3b = Me | R4 = Me; |
| (5) | R1 = 4-Cl-Ph | R2 = H | R3a = H | R3b = Me | R4 = Me; |
| (6) | R1 = 1-Napth | R2 = H | R3a = H | R3b = Me | R4 = Me; |
| (7) | R1 = Ph | R2 = H | R3a = H | R3b = Me | R4 = Me; |
| (8) | R1 = Ph | R2 = H | R3a = H | R3b = iPr | R4 = Me. |
| (9) | R1 = Ph | R2 = H | R3a = H | R3b = H | R4 = CH₃; |
| (10) | R1 = Ph | R2 = H | R3a = Me | R3b = Me | R4 = Me; |
| (11) | R1 = Ph | R2 = H | R3a = Me | R3b = H | R4 = Me; |
| (12) | R1 = Ph | R2 = H | R3a = H | R3b = CH₂Ph | R4 = Me; |
| (13) | R1 = Ph | R2 = H | R3a = CH₂Ph | R3b = H | R4 = Me; |
| (14) | R1 = Ph | R2 = H | R3a = iPr | R3b = H | R4 = Me; |
| (15) | R1 = Ph | R2 = H | R3a = H | R3b = Me | R4 = t-Bu; |
| (16) | R1 = Ph | R2 = H | R3a = H | R3b = Me | R4 = CH₂Ph; |
| (17) | R1 = 4-Me-Ph | R2 = H | R3a = H | R3b = Me | R4 = CH₃; |
| (18) | R1 = 4-Propyl-Ph | R2 = H | R3a = H | R3b = Me | R4 = Me; |
| (19) | R1 = 4-Neopent-Ph | R2 = H | R3a = H | R3b = Me | R4 = Me; |
| (20) | R1 = 4-MeO-Ph | R2 = H | R3a = H | R3b = Me | R4 = Me; |
| (21) | R1 = 4-CN-Ph | R2 = H | R3a = H | R3b = Me | R4 = Me; |
| (22) | R1 = 4-Br-Ph | R2 = H | R3a = H | R3b = Me | R4 = CH₂Ph; |
| (23) | R1 = 2-Cl-Ph | R2 = H | R3a = H | R3b = Me | R4 = Me; |
| (24) | R1 = 4-Cl-Ph | R2 = H | R3a = H | R3b = Me | R4 = CH₂Ph; |
| (25) | R1 = 2-Allyl-Ph | R2 = H | R3a = H | R3b = Me | R4 = Me; |
| (26) | R1 = 1-Napth | R2 = H | R3a = Me | R3b = Me | R4 = Me; |
| (27) | R1 = C₁₆H₃₃O(CH₂)₃ | R2 = H | R3a = H | R3b = H | R4 = Me; |
| (28) | R1 = C₁₆H₃₃O(CH₂)₃ | R2 = H | R3a = H | R3b = Me | R4 = Me; |
| (29) | R1 = C₁₆H₃₃O(CH₂)₃ | R2 = H | R3a = H | R3b = iPr | R4 = Me; |
| (30) | R1 = C₁₈H₃₇O(CH₂)₂ | R2 = H | R3a = H | R3b = Me | R4 = Me; and |
| (31) | R1 = Oleyl | R2 = H | R3a = H | R3b = Me | R4 = Me. |
